(19) United States
(12) Patent Application Publication
(10) Pub. No.: US 2019/0157571 A1
(43) Pub. Date: May 23, 2019

(54) COMPOUND, ORGANIC EL ELEMENT, DISPLAY DEVICE, AND ILLUMINATION DEVICE

(71) Applicant: Sharp Kabushiki Kaisha, Sakai City, Osaka (JP)

(72) Inventors: Tokiyoshi UMEDA, Sakai City (JP); Shigeru AOMORI, Sakai City (JP)

(21) Appl. No.: 16/300,614

(22) PCT Filed: Jun. 23, 2017
(86) PCT No.: PCT/JP2017/023126

§ 371 Date: Nov. 12, 2018

(30) Foreign Application Priority Data
Jun. 23, 2016 (JP) 2016-124402

(51) Int. Cl. H01L 51/00; C07D 209/86; C09K 11/06

(52) U.S. Cl. CPC H01L 51/0072; H01L 51/5012; C09K 11/06; C07D 209/86

(57) ABSTRACT

A compound represented by the following general formula (1): where R¹ is a hydrogen atom, a fluorine atom, an alkyl group, an alkoxy group, a fluorinated alkyl group, a cyano group, or a triphenylsilyl group, plural R¹ moieties may be the same as or different from one another and only any one of the plural R¹ moieties is a cyano group, R² is a hydrogen atom, a fluorine atom, an alkyl group, an alkoxy group, or a fluorinated alkyl group, plural R² moieties may be the same as or different from one another, and at least one of the R² moieties is a fluorinated alkyl group, R³ is a hydrogen atom, a fluorine atom, an alkyl group, an alkoxy group, a phenyl group, a carbazole group, a diphenylamino group, or a triphenylsilyl group, and plural R³ moieties may be the same as or different from one another.

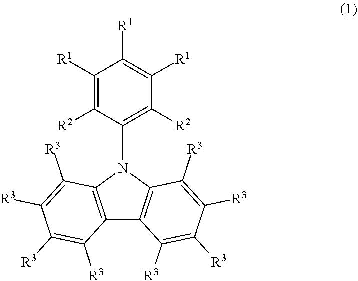

### TECHNICAL FIELD

Some aspects of the disclosure relate to a compound, an organic EL element, a display device, and an illumination device.

This application claims priority from JP 2016-124402 filed on Jun. 23, 2016 in Japan, the entirety of which is incorporated by reference herein.

### BACKGROUND ART

In the related art, a display (an organic EL display and a display device) using an organic EL element has been developed. Since the organic EL display does not need a backlight, the organic EL display has an advantage of enabling a thickness reduction and power-saving as compared to a display using a liquid crystal panel.

The organic EL element includes a pair of electrodes, and function layers such as a light-emitting layer, an electron transport layer, and a hole transport layer interposed between the pair of electrodes. In recent years, to obtain a high-performance organic EL element, a novel compound constituting each of the function layers has been discussed (see, for example, PTL 1 and PTL 2).

### CITATION LIST

### Patent Literature

PTL 1: WO 2013/154064

PTL 2: WO 2015/008580

### SUMMARY

### Technical Problem

On the other hand, a currently known organic EL display still need to be improved in terms of luminous efficiency. Thus, a novel compound capable of achieving an organic EL display having desired performance has been in demand.

Some aspects of the disclosure have been made under such a circumstance, and the disclosure aims to provide a novel compound improving luminous efficiency and capable of producing a high-performance organic EL element. Moreover, the disclosure aims to provide an organic EL element using such a compound and having improved luminous efficiency. Moreover, the disclosure aims to provide a display device and an illumination device each using such an organic EL element.

### Solution to Problem

According to a first aspect of the disclosure, a compound represented by the following general formula (1) is provided.

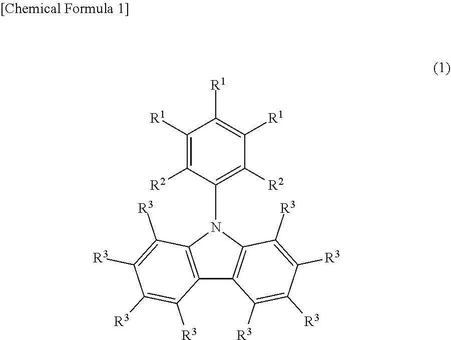

where R¹ is a hydrogen atom, a fluorine atom, an alkyl group having from 1 to 22 carbons, an alkoxy group having from 1 to 22 carbons, a fluorinated alkyl group having from 1 to 22 carbons, a cyano group, or a triphenylsilyl group, plural R¹ moieties may be the same as or different from one another, and only any one of the plural R¹ moieties is a cyano group, R² is a hydrogen atom, a fluorine atom, an alkyl group having from 1 to 22 carbons, an alkoxy group having from 1 to 22 carbons, or a fluorinated alkyl group having from 1 to 22 carbons, plural R² moieties may be the same as or different from one another, and at least one of the R² moieties is a fluorinated alkyl group having from 1 to 22 carbons, and R³ is a hydrogen atom, a fluorine atom, an alkyl group having from 1 to 22 carbons, an alkoxy group having from 1 to 22 carbons, a phenyl group, a carbazole group, a diphenylamino group, or a triphenylsilyl group, and plural R³ moieties may be the same as or different from one another.

According to a second aspect of the disclosure, an organic EL element including a layer including the compound described above is provided.

According to a third aspect of the disclosure, a display device including a first organic EL element configured to emit red light, a second organic EL element configured to emit green light, and a third organic EL element configured to emit blue light is provided, and in the display device, at least any one of the first organic EL element, the second organic EL element, and the third organic EL element is the organic EL element described above.

According to a fourth aspect of the disclosure, a display device including the organic EL element described above, and a phosphor layer configured to absorb light emitted from the organic EL element to fluoresce is provided.

According to a fifth aspect of the disclosure, a display device including the organic EL element described above, and a color filter layer configured to convert white light emitted from the organic EL element into at least one kind of red light, green light, and blue light is provided.

According to a sixth aspect of the disclosure, an illumination device including the organic EL element described above and configured to emit white light is provided.

### Advantageous Effects of Disclosure

According to some aspects of the disclosure, a novel compound improving luminous efficiency and capable of producing a high-performance organic EL element can be provided. A compound according to an aspect of the disclosure includes a compound radiating thermally activated delayed fluorescence. Moreover, an organic EL element, a display device, and an illumination device each using such a compound and having improved luminous efficiency can be provided.

### BRIEF DESCRIPTION OF DRAWINGS

FIG. 1 is a schematic view illustrating an organic EL element according to an aspect of the disclosure.

FIG. 2 is a schematic cross-sectional view of a display device according to a third embodiment.

FIG. 3 is a plan view illustrating a main portion of the display device according to the third embodiment.

FIG. 4 is an equivalent circuit diagram illustrating the main portion of the display device according to the third embodiment.

FIG. 5 is a schematic cross-sectional view of a display device according to a fourth embodiment.

FIG. 6 is a schematic cross-sectional view of a display device according to a fifth embodiment.

FIG. 7 is a schematic view of an electronic device according to a sixth embodiment.

FIG. 8 is a schematic view of an electronic device according to the sixth embodiment.

FIG. 9 is a schematic view of an electronic device according to the sixth embodiment.

FIG. 10 shows a ¹H-NMR spectrum of compound (101) of Example 1.

FIG. 11 shows an absorption spectrum, a fluorescence spectrum, and a phosphorescence spectrum of compound (101) of Example 1.

FIG. 12 shows an emission decay curve of compound (101) of Example 1.

FIG. 13 shows a time-resolved emission spectrum of compound (101) of Example 1.

FIG. 14 shows a ¹H-NMR spectrum of compound (102) of Example 2.

FIG. 15 shows an absorption spectrum, a fluorescence spectrum, and a phosphorescence spectrum of compound (102) of Example 2

FIG. 16 shows an emission decay curve of compound (102) of Example 2.

FIG. 17 shows a time-resolved emission spectrum of compound (102) of Example 2.

FIG. 18 shows a ¹H-NMR spectrum of compound (103) of Example 3.

FIG. 19 shows an absorption spectrum, a fluorescence spectrum, and a phosphorescence spectrum of compound (103) of Example 3.

FIG. 20 shows an emission decay curve of compound (103) of Example 3.

FIG. 21 shows a time-resolved emission spectrum of compound (103) of Example 3.

FIG. 22 shows an absorption spectrum, a fluorescence spectrum, and a phosphorescence spectrum of compound (201) of Comparative Example 1.

FIG. 23 shows an emission decay curve of compound (201) of Comparative Example 1.

FIG. 24 shows a time-resolved emission spectrum of compound (201) of Comparative Example 1.

### DESCRIPTION OF EMBODIMENTS

### First Embodiment

### Compound

A compound according to an aspect of the disclosure is represented by the following general formula (1). Hereinafter, the compound represented by the following formula (1) may be referred to as “compound (1)”. Compound (1) is a novel compound.

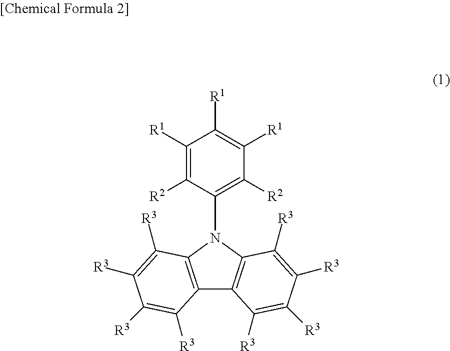

where R¹ is a hydrogen atom, a fluorine atom, an alkyl group having from 1 to 22 carbons, an alkoxy group having from 1 to 22 carbons, a fluorinated alkyl group having from 1 to 22 carbons, a cyano group, or a triphenylsilyl group, plural R¹ moieties may be the same as or different from one another, and only any one of the plural R¹ moieties is a cyano group,

R² is a hydrogen atom, a fluorine atom, an alkyl group having from 1 to 22 carbons, an alkoxy group having from 1 to 22 carbons, or a fluorinated alkyl group having from 1 to 22 carbons, plural R² moieties may be the same as or different from one another, and at least one of the R² moieties is a fluorinated alkyl group having from 1 to 22 carbons, and

R³ is a hydrogen atom, a fluorine atom, an alkyl group having from 1 to 22 carbons, an alkoxy group having from 1 to 22 carbons, a phenyl group, a carbazole group, a diphenylamino group, or a triphenylsilyl group, and plural R³ moieties may be the same as or different from one another.

That is, compound (1) includes a structure in which a benzene ring is bonded to a nitrogen atom of carbazole having a donor property. Further, compound (1) includes a structure in which a cyano group being an electron withdrawing substituent and a fluorinated alkyl group also being an electron withdrawing substituent are bonded to each other on a benzene ring.

In the formula, the R¹ is a hydrogen atom, a fluorine atom, an alkyl group having from 1 to 22 carbons, an alkoxy group having from 1 to 22 carbons, a fluorinated alkyl group having from 1 to 22 carbons, a cyano group, or a triphenylsilyl group.

The alkyl group in the R¹ may be any of linear, branched, and cyclic, and in a case where the alkyl group is cyclic, the alkyl group may be any of monocyclic and polycyclic. The alkyl group preferably has from 1 to 18 carbons, more preferably from 1 to 10 carbons, and still more preferably from 1 to 6 carbons.

Examples of the alkyl group having from 1 to 22 carbons can include a methyl group, an ethyl group, an n-propyl group, an isopropyl group, an n-butyl group, an isobutyl group, a sec-butyl group, a tert-butyl group, an n-pentyl group, an isopentyl group, a neopentyl group, a tert-pentyl group, a 1-methylbutyl group, an n-hexyl group, a 2-methylpentyl group, a 3-methylpentyl group, a 2,2-dimethylbutyl group, a 2,3-dimethylbutyl group, an n-heptyl group, a 2-methylhexyl group, a 3-methylhexyl group, a 2,2-dimethylpentyl group, a 2,3-dimethylpentyl group, a 2,4-dimethylpentyl group, a 3,3-dimethylpentyl group, a 3-ethylpentyl group, a 2,2,3-trimethylbutyl group, an n-octyl group, an isooctyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an icosyl group, a heneicosyl group, and a docosyl group.

The cyclic alkyl group preferably has from 3 to 22 carbons, and examples of such an alkyl group can include a cyclopropyl group, a cyclobutyl group, a cyclopentyl group, a cyclohexyl group, a cycloheptyl group, a cyclooctyl group, a cyclononyl group, a cyclodecyl group, a norbornyl group, an isobornyl group, a 1-adamantyl group, a 2-adamantyl group and a tricyclodecyl group, and can further include these cyclic alkyl groups each having one or more hydrogen atoms substituted by a linear, branched, or cyclic alkyl group. Here, examples of the linear, branched, and cyclic alkyl groups substituting the hydrogen atoms include those groups described above as the examples of the alkyl group in the R¹.

Examples of the alkoxy group in the R¹ can include a monovalent group such as a methoxy group and a cyclopropoxy group in which the above-described alkyl group is bonded to an oxygen atom. The alkyl group that the alkoxy group includes may be any of linear, branched, and cyclic, and in a case where the alkyl group is cyclic, the alkyl group may be any of monocyclic and polycyclic. The alkyl group that the alkoxy group includes preferably has from 1 to 18 carbons, more preferably from 1 to 10 carbons, and still more preferably from 1 to 6 carbons.

An example of the fluorinated alkyl group in the R¹ can include the above-described alkyl group having one or more hydrogen atoms substituted by a fluorine atom.

The three R¹ moieties of compound (1) may be the same as or different from one another. Moreover, only any one of the R¹ moieties is a cyano group and the remaining two R¹ moieties are groups other than the cyano group among the above-described substituents.

In the formula, the R² is a hydrogen atom, a fluorine atom, an alkyl group having from 1 to 22 carbons, an alkoxy group having from 1 to 22 carbons, or a fluorinated alkyl group having from 1 to 22 carbons. Examples of the alkyl group, the alkoxy group, and the fluorinated alkyl group in the R² can include the same as the examples of the alkyl group, the alkoxy group, and the fluorinated alkyl group in the R¹.

The two R² moieties of compound (1) may be the same as or different from each other. Moreover, at least one of the R² moieties is a fluorinated alkyl group having from 1 to 22 carbons.

In the formula, the R³ is a hydrogen atom, a fluorine atom, an alkyl group having from 1 to 22 carbons, an alkoxy group having from 1 to 22 carbons, a phenyl group, a carbazole group, a diphenylamino group, or a triphenylsilyl group. Examples of the alkyl group and the alkoxy group in the R³ can include the same as the examples of the alkyl group and the alkoxy group in the R¹.

The eight R³ moieties of compound (1) may be the same as or different from one another.

Note that although the examples of the R¹, R² and R³ of compound (1) are described above, the substituents usable as the R¹, R², and R³ are not limited to those described above, and other substituents can also be adopted.

In compound (1), one of the two R² moieties is preferably a hydrogen atom. That is, a preferable structure of a compound according to an aspect of the disclosure is represented by the following general formula (2). Hereinafter, the compound represented by the following formula (2) may be referred to as “compound (2)”.

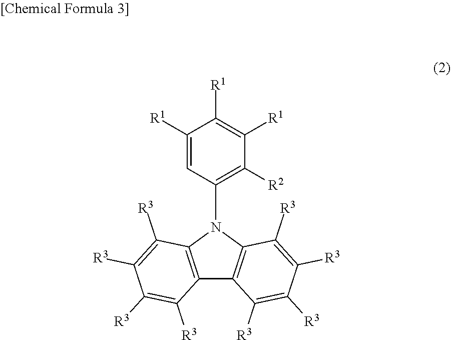

where R¹ and R³ are the same as those in formula (1), and

R² is a fluorinated alkyl group having from 1 to 22 carbons.

Moreover, in compound (2), two of three R¹ moieties are preferably hydrogen atoms, and the R¹ opposed to a carbon atom bonded to a nitrogen atom of a carbazole group (bonded to a carbon atom present at a para position with respect to the carbon atom bonded to the nitrogen atom) is preferably a cyano group.

Further, among eight R³ moieties in a carbazole skeleton in compound (2), the six R³ moieties present at 1st, 2nd, 4th, 5th, 7th, and 8th positions are preferably hydrogen atoms, and the two R³ moieties bonded to carbon atoms present at 3rd and 6th positions are preferably the substituents described above as the examples of the R³.

That is, a preferable structure of a compound according to an aspect of the disclosure is represented by the following general formula (3). Hereinafter, the compound represented by the following formula (3) may be referred to as “compound (3)”.

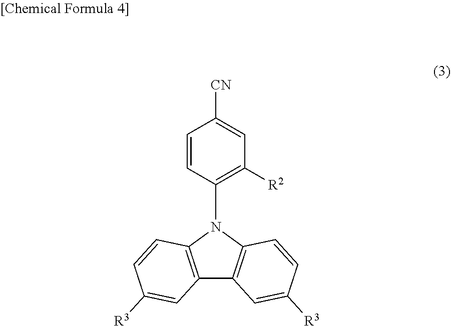

where R² is a fluorinated alkyl group having from 1 to 22 carbons, and

R³ is the same as in formula (1).

The R³ is used for adjusting entirely a molecular weight of each of compounds (1) to (3). That is, to decrease the molecular weight of each of compounds (1) to (3), the hydrogen atom, or the alkyl group and the alkoxy group each having a small number of carbons may be selected from among the substituents described above as the groups usable for the R³. When the molecular weights of compounds (1) to (3) are small, compounds (1) to (3) become soluble in a solvent, and become coatable as a solution.

Moreover, to increase the molecular weight of each of compounds (1) to (3), the alkyl group and the alkoxy group each having a large number of carbons, the phenyl group, the carbazole group, the diphenylamino group, or the triphenylsilyl group may be selected from among the substituents described above as the groups usable for the R³. Among these, the substituent such as the phenyl group and the carbazole group is conjugated with a conjugated system of a main skeleton of each of compounds (1) to (3) and thus, such a substituent is considered to affect a luminescence wavelength in use of each of compounds (1) to (3) as a luminescent material. Thus, a luminescence wavelength in use of each of compounds (1) to (3) as a luminescent material can be adjusted by appropriately selecting the R³.

Examples of compounds (1) to (3) can include the following formulae (11) to (26). The following formulae (11) to (26) are described as examples, and compounds (1) to (3) are not limited to these examples.

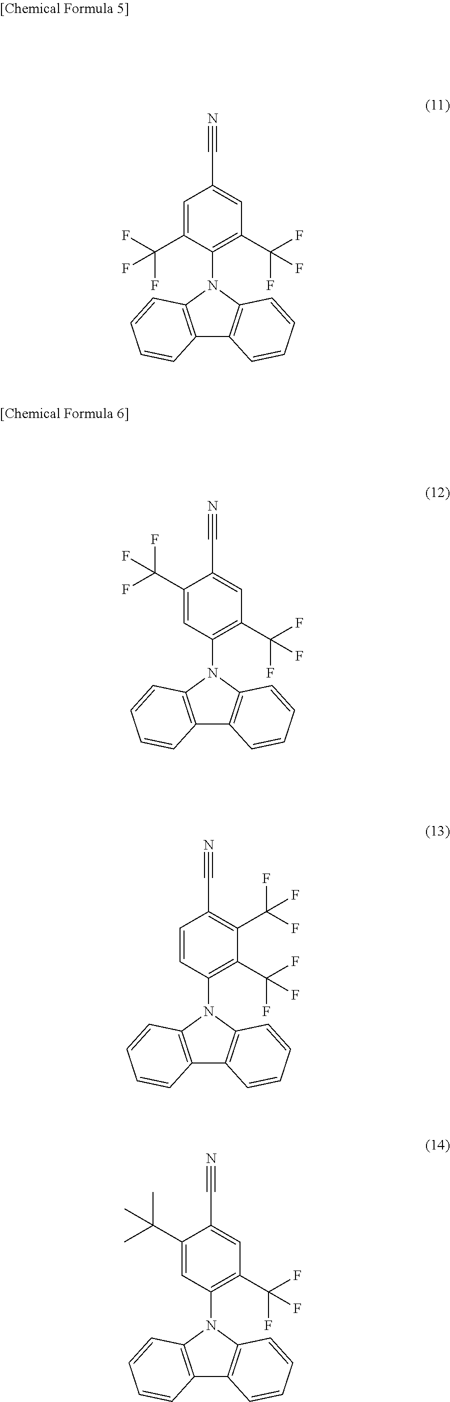 [SMILES: N#Cc1cc(C(F)(F)F)c(-n2c3ccccc3c3ccccc32)c(C(F)(F)F)c1 ; N#Cc1cc(C(F)(F)F)c(-n2c3ccccc3c3ccccc32)cc1C(F)(F)F ; N#Cc1ccc(-n2c3ccccc3c3ccccc32)c(C(F)(F)F)c1C(F)(F)F ; CC(C)(C)c1cc(-n2c3ccccc3c3ccccc32)c(C(F)(F)F)cc1C#N]

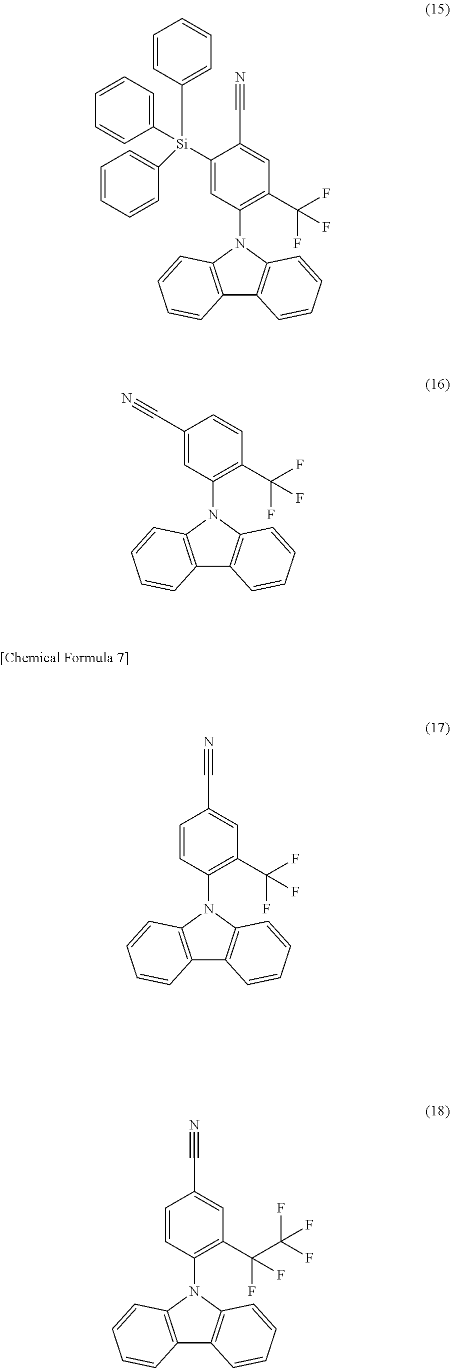 [SMILES: N#Cc1cc(C(F)(F)F)c(-n2c3ccccc3c3ccccc32)cc1[Si](c1ccccc1)(c1ccccc1)c1ccccc1 ; N#Cc1ccc(C(F)(F)F)c(-n2c3ccccc3c3ccccc32)c1 ; N#Cc1ccc(-n2c3ccccc3c3ccccc32)c(C(F)(F)F)c1 ; N#Cc1ccc(-n2c3ccccc3c3ccccc32)c(C(F)(F)C(F)(F)F)c1]

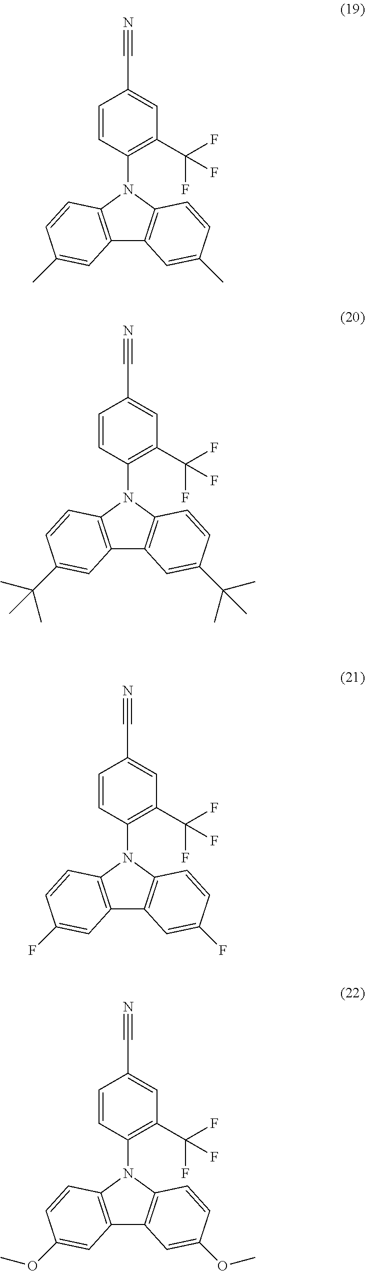

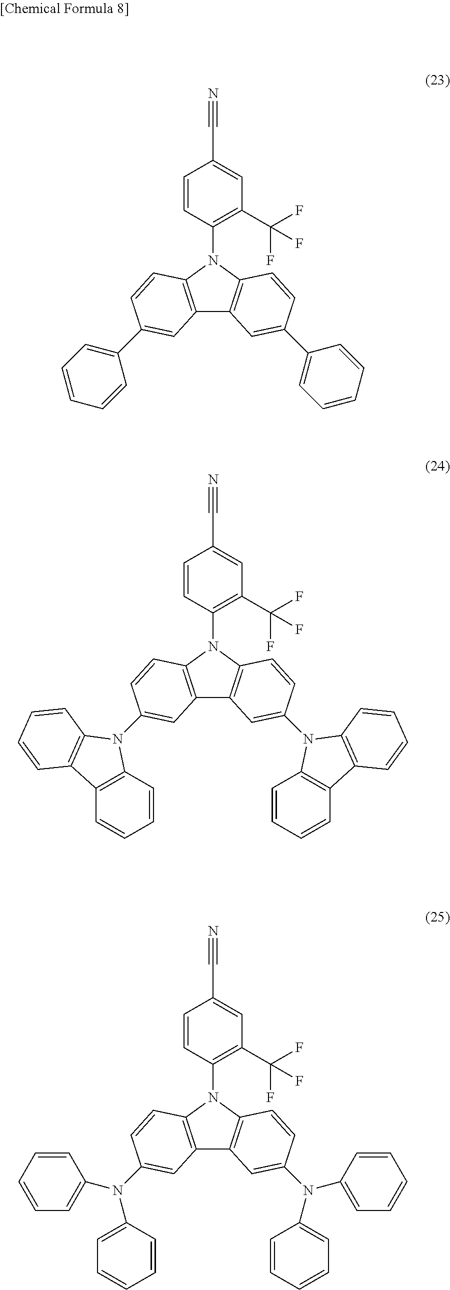 [SMILES: N#Cc1ccc(-n2c3ccc(-c4ccccc4)cc3c3cc(-c4ccccc4)ccc32)c(C(F)(F)F)c1 ; N#Cc1ccc(-n2c3ccc(-n4c5ccccc5c5ccccc54)cc3c3cc(-n4c5ccccc5c5ccccc54)ccc32)c(C(F)(F)F)c1 ; N#Cc1ccc(-n2c3ccc(N(c4ccccc4)c4ccccc4)cc3c3cc(N(c4ccccc4)c4ccccc4)ccc32)c(C(F)(F)F)c1]

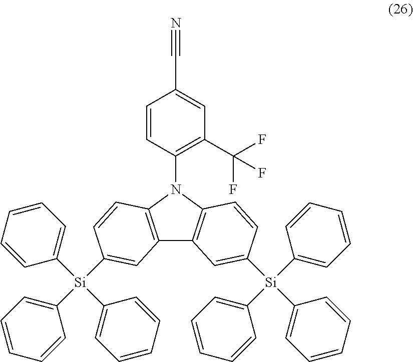 [SMILES: N#Cc1ccc(-n2c3ccc([Si](c4ccccc4)(c4ccccc4)c4ccccc4)cc3c3cc([Si](c4ccccc4)(c4ccccc4)c4ccccc4)ccc32)c(C(F)(F)F)c1]

Compounds (1) to (3) described above each include an “intramolecular donor-acceptor type” structure including an acceptor benzene skeleton and a donor carbazole skeleton. The acceptor benzene skeleton has an electron-withdrawing cyano group as the R¹ and has an electron-withdrawing fluorinated alkyl group as the R².

Moreover, compounds (1) to (3) each include a structure where a plane of the benzene skeleton and a plane of the carbazole group are twisted largely in a vertical direction at a junction of the benzene skeleton and the carbazole group owing to steric hindrance of the fluorinated alkyl group and the carbazole group in the R².

Accordingly, compounds (1) to (3) each have an electronic state in which an energy level difference (ΔE_{ST}) between a singlet excited state (S₁) and a triplet excited state (T₁) is small.

Thus, in use of each of compounds (1) to (3) as a luminescent material, thermally activated delayed fluorescence is exhibited. Use of each of such compounds (1) to (3) as a luminescent material of an organic EL element can provide a light emitting element having good luminous efficiency (external quantum yield) of the organic EL element and thus, such use of compounds (1) to (3) is preferable.

Compounds (1) to (3) described above can be produced by causing corresponding fluorobenzonitriles to react with carbazoles. As an example, compound (17) described above can be produced by the following reaction scheme.

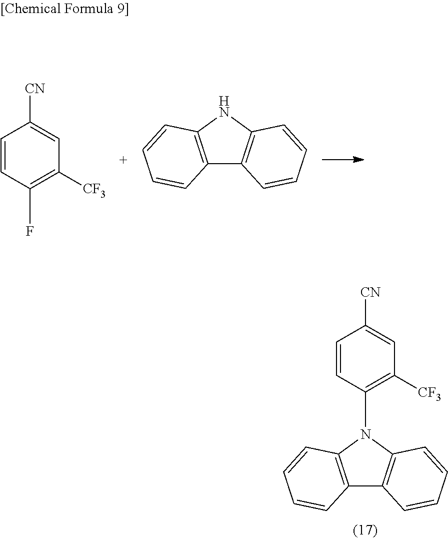 [SMILES: N#Cc1ccc(F)c(C(F)(F)F)c1 ; c1ccc2c(c1)[nH]c1ccccc12 ; N#Cc1ccc(-n2c3ccccc3c3ccccc32)c(C(F)(F)F)c1]

A structure of a product in the reaction scheme described above can be confirmed by using known technique such as Nuclear Magnetic Resonance (NMR) spectroscopy, Mass Spectrometry (MS), Infrared Spectroscopy (IR), and ultraviolet-visible spectroscopy (UV-VIS absorption spectrum).

According to the above-described compounds, a novel compound improving luminous efficiency and capable of producing a high-performance organic EL element can be provided.

### Second Embodiment

### Organic EL Element

In an organic EL element, carriers are injected from both anode and cathode electrodes, and a luminescent material is brought into an excited state to emit light. Generally, an excited singlet state occupies 25% and an excited triplet state occupies 75% of the excited state (excitons) generated by the carrier injection.

Of the above-described excited state, the excited triplet state has a lifetime longer than a lifetime of the excited singlet state. Thus, energy of an organic EL material in the excited triplet state is readily quenched, for example, owing to saturation of the excited state, an interaction between excitons in the excited triplet state, or energy transfer to oxygen, impurities, and deteriorated molecules. Owing to this fact, generally phosphorescence that is light emission from the excited triplet state frequently does not have a high photoluminescence quantum yield.

On the other hand, in the case of a material exhibiting delayed fluorescence (hereinafter may be referred to as a delayed fluorescent material), for example, a material in the excited triplet state absorbs thermal energy and thus, the material undergoes inverse intersystem crossing from the excited triplet state to the excited singlet state. The material brought into the excited singlet state radiates fluorescence.

In an organic EL element, a thermally activated delayed fluorescent material due to thermal energy absorption is considered particularly useful. When the thermally activated delayed fluorescent material is used for an organic EL element, excitons in the excited singlet state occupying 25% and generated by the carrier injection radiate prompt fluorescence. On the other hand, excitons in the excited triplet state occupying 75% and generated by the carrier injection absorb heat at room temperature, heat generated by a device, or the like and undergo inverse intersystem crossing from the excited triplet state to the excited singlet state to radiate delayed fluorescence.

At this time, since the prompt fluorescence and the delayed fluorescence are both light emission from the excited singlet state, the prompt fluorescence and the delayed fluorescence are of the same spectral shape. However, since a delay occurs owing to the inverse intersystem crossing from the excited triplet state to the excited singlet state, a lifetime of the delayed fluorescence via the excited triplet state is longer than a lifetime of the prompt fluorescence.

Use of such a thermally activated delayed fluorescent material can increase a ratio of the excited singlet state to 25% or greater, and in principle, up to 100%, while the ratio of the excited singlet state produced in an ordinary fluorescent material is only 25%. Moreover, a compound radiating intense fluorescence and delayed fluorescence even at low temperature of less than 100° C. can sufficiently undergo inverse intersystem crossing from the excited triplet state to the excited singlet state owing to heat at room temperature, heat generated by a device, or the like and can radiate delayed fluorescence. Thus, use of such a delayed fluorescent material as a material for forming an organic EL element can drastically improve luminous efficiency.

As described above, the compound according to an aspect of the disclosure exhibits thermally activated delayed fluorescence. Therefore, use of the compound according to an aspect of the disclosure as a luminescent material of a light-emitting layer can provide an excellent organic EL element.

FIG. 1 is a schematic view illustrating an organic EL element according to an aspect of the disclosure. An organic EL element 100 according to an aspect of the disclosure includes an anode electrode 2, a cathode electrode 3, and a light-emitting layer 4 interposed between the anode electrode 2 and the cathode electrode 3. Moreover, the organic EL element 100 includes, as optional constituent elements, a substrate 1, a hole transport layer 5, and an electron transport layer 6. In the organic EL element 100, for example, the substrate 1, the anode electrode 2, the hole transport layer 5, the light-emitting layer 4, the electron transport layer 6, and the cathode electrode 3 are layered one on another in this order. Each of the layers may be a single layer or may include a configuration in which a plurality of layers are layered one on another. Moreover, a hole injection layer, an electron blocking layer, a hole blocking layer, an electron injection layer, an exciton blocking layer, and the like may be layered appropriately. The hole transport layer may be a hole injection transport layer having a hole injecting function, and the electron transport layer may be an electron injection transport layer having an electron injecting function.

The substrate 1 can be formed by using any of a material having optical transparency and a material having no optical transparency, as long as the anode electrode 2 is formed on the substrate 1 and the substrate 1 is capable of supporting a layer structure of the organic EL element. For example, a material such as glass, plastic, quartz, and silicon can be used as the material for forming the substrate 1. Moreover, the substrate 1 is provided with a driving TFT element configured to drive the organic EL element.

The anode electrode 2 can be formed of a conductive material having a large work function (for example, 4 eV or greater; where 1 eV=1.602×10⁻¹⁹ J). As such a material, a metal, an alloy, a conductive metal compound, and a mixture of these materials can be used. Specific examples of such a material include a metal such as Au, a conductive metal oxide such as Indium Tin Oxide (ITO), SnO₂ and ZnO, and CuI.

The anode electrode 2 can be obtained, for example, by forming a thin film made of the material for forming the anode electrode 2 on the substrate 1 by PVD or CVD, and then forming the thin film into a desired pattern by a photolithography method.

The cathode electrode 3 can be formed of a conductive material having a small work function (for example, less than 4 eV). As such a material, a metal, an alloy, a conductive metal compound, and a mixture of these materials can be used. Specific examples of such a material include sodium, a sodium-potassium alloy, magnesium, lithium, a magnesium/copper mixture, a magnesium/silver mixture, a magnesium/aluminum mixture, a magnesium/indium mixture, an aluminum/aluminum oxide (Al₂O₃) mixture, indium, a lithium/aluminum mixture, and a rare earth metal.

The cathode electrode 3 can be formed, for example, by Physical Vapor Deposition (PVD) such as vapor deposition and sputtering.

The light-emitting layer 4 is a layer configured to radiate light having a wavelength corresponding to an energy gap of a luminescent material owing to excitation of the luminescent material due to holes injected from the anode electrode and electrons injected from the cathode electrode, and further owing to coupling of the holes and the electrons. The light-emitting layer 4 may be formed by using a luminescent material alone or may include the luminescent material and a host material.

In a case where the compound according to an aspect of the disclosure is used as a luminescence dopant or an assist dopant, a host molecule known as a host molecule of an organic EL element can be used as a host molecule in this case.

Examples of the host molecule that can be used include a carbazole derivative such as 1,3-bis(N-carbazolyl)benzene (mCP), 9,9′-(2,6-pyridinediyl)bis-9H-carbazole (PYD2), 4,4′-bis(N-carbazolyl)-1,1′-biphenyl (CBP), 9,9′-biphenyl-3,3′-diylbis-9H-carbazole (mCBP), 4,4′-bis(9-carbazolyl)-2,2′-dimethylbiphenyl (CDBP), 9,9-di(4-dicarbazole-benzyl)fluorene (CPF), and poly(N-octyl-2,7-carbazole-O-9,9-dioctyl-2,7-fluorene) (PCF); an aniline derivative such as 4-(diphenylphosphoyl)-N,N-diphenylaniline (HM-A1); a fluorene derivative such as 1,3-bis(9-phenyl-9H-fluoren-9-yl)benzene (mDPFB), 1,4-bis(9-phenyl-9H-fluoren-9-yl)benzene (pDPFB); 1,3,5-tris[4-(diphenylamino)phenyl]benzene (TDAPB), polyvinyl carbazole (PVK), 1,4-bis(triphenylsilyl)benzene (UGH2), and bis[2-(diphenylphosphino)phenyl]ether oxide (DPEPO).

The host molecule used in the case where the compound according to an aspect of the disclosure is used as a luminescence dopant or an assist dopant is not limited to the host molecules described above. The host material in the light-emitting layer is preferably an organic compound having a hole transporting ability and an electron transporting ability, and preventing light emission at a long wavelength, and having a high glass transition temperature.

Moreover, in a case where the compound according to an aspect of the disclosure is used as a host molecule or an assist dopant, a known luminescent material for an organic EL element can be used as a luminescence dopant in this case.

Examples of the luminescence dopant that can be used include a fluorescent material such as a coumarin-type dye, a pyran-type dye, a cyanine-type dye, a croconium-type dye, a squarylium-type dye, an oxobenzanthracene-type dye, a fluorescein-type dye, a rhodamine-type dye, a pyrylium-type dye, a perylene-type dye, a stilbene-type dye, and a polythiophene-type dye, or a rare earth complex luminescent material, and a compound having a high fluorescent quantum yield such as, typically, a laser dye. Moreover, examples of the luminescence dopant also include, but not limited to, an Ir complex of a phosphorescent material such as tris(2-phenylpyridine)iridium (III) (Ir(ppy)₃), Ir(piq)₃, Ir(ppy)₂(acac), Ir(piq)₂ acac, Ir(Btp)₂ acac, bis[(4,6-difluorophenyl)-pyridinato-N, C2′]picolinate iridium (III) (FIrpic), bis(4′,6′-difluorophenylpolydinato)tetrakis(1-pyrazoyl)borate iridium (III) (FIr6), tris[N-(4′-cyanophenyl)-N′-methylimidazol-2-ylidene-C2,C2′]iridium (III) (Ir(cn-pmic)₃), tris((3,5-difluoro-4-cyanophenyl)pyridine)iridium (FCNIr), Ir(dbfmi), Ir(fbppz)₂(dfbdp), FIrN₄, or Ir(cnbic)₃; a Cu complex such as [Cu(dnbp)(DPEPhos)]BF₄, [Cu(dppb)(DPEPhos)]BF₄, [Cu(μ-1)dppb]₂, [(Cu(μ-C1)DPEPhos)]₂, Cu(2-tzq)(DPEPhos), [Cu(PNP)]₂, compound 1001, and Cu(Bpz₄)(DPEPhos); a Pt complex such as FPt and Pt-4; a complex of a heavy atom metal such as rhenium (Re), ruthenium (Ru), copper (Cu), or osmium (Os); and variety of metal complexes known in the related art.

The hole transport layer 5 is an optional constituent element in the organic EL element 100, and is a layer having a function to transport holes. The hole transport layer can be provided as a single layer or a plurality of layers. A material capable of any of hole injection or transport, and electron blocking can be used as a hole transport material. A hole transport material known as a hole transport material of an organic EL element can be used as the hole transport material.

Examples of the hole transport material that can be used include a carbazole derivative, a triazole derivative, an oxadiazole derivative, an imidazole derivative, an indolocarbazole derivative, a polyarylalkane derivative, a pyrazoline derivative and a pyrazolone derivative, a phenylenediamine derivative, an arylamine derivative, an amino substituted chalcone derivative, an oxazole derivative, a styrylanthracene derivative, a fluorenone derivative, a hydrazone derivative, a stilbene derivative, a silazane derivative, a porphyrin compound, an aniline type copolymer, and a conductive high molecular oligomer, and particularly a thiophene oligomer. Particularly, an aromatic tertiary amine compound and a styrylamine compound are preferably used, and the aromatic tertiary amine compound is more preferably used, but the hole transport material is not limited to these compounds.

The electron transport layer 6 is an optional constituent element in the organic EL element 100, and is a layer having a function to transport electrons. The electron transport layer can be provided as a single layer or a plurality of layers. A material capable of any of hole injection or transport, and electron blocking can be used as an electron transport material. A material known as an electron transport material of an organic EL element can be used as the electron transport material.

Examples of the electron transport material that can be used include an oxadiazole derivative, a nitro-substituted fluorene derivative, a diphenylquinone derivative, a thiopyran dioxide derivative, a carbodiimide derivative, a fluorenylidenemethane derivative, and an anthraquinodimethane derivative, and an anthrone derivative. Further, in the oxadiazole derivative, a thiadiazole derivative having an oxygen atom of an oxadiazole ring substituted by a sulfur atom, and a quinoxaline derivative having a quinoxaline ring known as an electron-withdrawing substituent can be used as the electron transport material. Further, a polymer material including these materials introduced into a polymer chain or a polymer material including these materials as a main chain of a polymer can also be used, but the electron transport material is not limited to these polymer materials.

The hole injection layer and the electron injection layer may be present between the anode electrode and the light-emitting layer or the hole transport layer and between the cathode electrode and the light-emitting layer or the electron transport layer, respectively. The injection layer can be provided, as necessary, between the electrode and the organic layer to lower a driving voltage or to improve light emission luminance.

The electron blocking layer or the hole blocking layer is a layer capable of blocking diffusion of charges (electrons or holes) and/or excitons present in the light-emitting layer to the outside of the light-emitting layer.

The electron blocking layer can be disposed between the light-emitting layer and the hole transport layer, and blocks traveling of electrons from the light-emitting layer to the hole transport layer. Similarly, the hole blocking layer can be disposed between the light-emitting layer and the electron transport layer, and blocks traveling of holes from the light-emitting layer to the electron transport layer.

Moreover, the blocking layers can be used to block diffusion of excitons to the outside of the light-emitting layer. That is, each of the electron blocking layer and the hole blocking layer can also function as an exciton blocking layer.

The electron blocking layer or the exciton blocking layer can be a single layer having functions of the electron blocking layer and the exciton blocking layer. The hole blocking layer has, in a broad sense, a function of the electron transport layer.

The hole blocking layer plays a role of transporting electrons and also blocking arrival of holes on the electron transport layer and accordingly, the hole blocking layer can improve a recombination probability of electrons and holes in the light-emitting layer. As a material of the hole blocking layer, a material of the electron transport layer can be used as necessary.

The electron blocking layer has, in a broad sense, a function to transport holes. The electron blocking layer plays a role of transporting holes and also blocking arrival of electrons on the hole transport layer and accordingly, the electron blocking layer can improve a probability of recombination of electrons and holes in the light-emitting layer.

The exciton blocking layer refers to a layer configured to block diffusion of excitons generated by recombination of holes and electrons in the light-emitting layer to a charge transport layer. The exciton blocking layer is inserted to enable efficient confinement of excitons in the light-emitting layer, and can improve luminous efficiency of the element. The exciton blocking layer can be inserted to any of the anode electrode side and the cathode electrode side to be adjacent to the light-emitting layer, and can also be inserted simultaneously to each of the anode electrode side and the cathode electrode side. That is, in a case where the exciton blocking layer is inserted to the anode electrode side, the exciton blocking layer can be inserted between the hole transport layer and the light-emitting layer, and in a case where the exciton blocking layer is inserted to the cathode electrode side, the exciton blocking layer can be inserted between the light-emitting layer and the cathode electrode. In each of the cases, the exciton blocking layer can be inserted to be adjacent to the light-emitting layer.

Moreover, between the anode electrode and the exciton blocking layer adjacent to the anode electrode side of the light-emitting layer, the hole injection layer, the electron blocking layer, or the like can be incorporated. Between the cathode electrode and the exciton blocking layer adjacent to the cathode electrode side of the light-emitting layer, the electron injection layer, the electron transport layer, the hole blocking layer, or the like can be incorporated. In a case where the blocking layer is disposed, at least any one kind of excited singlet energy and excited triplet energy of the material used as the blocking layer is preferably higher than a corresponding kind of excited singlet energy and excited triplet energy of the luminescent material.

Such an organic EL element according to an aspect of the disclosure includes the compounds described above as compounds (1) to (3) in any of the light-emitting layer 4, the hole transport layer 5, and the electron transport layer 6. Compounds (1) to (3) describe above are useful as a material for forming an organic EL element.

First, in a case where any of compounds (1) to (3) is incorporated as a material for forming the light-emitting layer 4, the following effects can be expected.

As described above, compounds (1) to (3) each include a delayed fluorescent material radiating delayed fluorescence. An organic EL element using the delayed fluorescent material as a luminescent material radiates delayed fluorescence, and has high luminous efficiency.

In the case where any of compounds (1) to (3) is used as the material for forming the light-emitting layer 4, these compounds may function as a luminescence dopant, or may function as an assist dopant, or may function as a host molecule, or may function as an exciplex molecule. In a case where compounds (1) to (3) exert these functions, known compounds used for the hole transport layer, the electron transport layer, the hole injection layer, the electron blocking layer, the hole blocking layer, the electron injection layer, the exciton blocking layer, or the like can be used as the pairs to compounds (1) to (3).

In the case of using a host molecule, a content of the compound according to an aspect of the disclosure as a luminescent material in the light-emitting layer is preferably 0.1 wt. % or greater, and more preferably 1 wt. % or greater.

Moreover, in the case of using a host molecule, a content of the compound according to an aspect of the disclosure as a luminescent material in the light-emitting layer is preferably 50 wt. % or less, more preferably 20 wt. % or less, and still more preferably 15 wt. % or less.

In the case of using a host molecule and a luminescence dopant and using the compound according to an aspect of the disclosure as an assist dopant, a content of the compound in the light-emitting layer is preferably 1 wt. % or greater, and more preferably 5 wt. % or greater. Moreover, in the case of using a host molecule and a luminescence dopant and using the compound according to an aspect of the disclosure as an assist dopant, a content of the compound in the light-emitting layer is preferably 50 wt. % or less, more preferably 30 wt. % or less, and still more preferably 20 wt. % or less.

In the case of using a luminescence dopant, a content of the compound according to an aspect of the disclosure as a host molecule in the light-emitting layer is preferably 70 wt. % or greater, and more preferably 85 wt. % or greater. Moreover, in the case of using a luminescence dopant, a content of the compound according to an aspect of the disclosure as a host molecule in the light-emitting layer is preferably 99 wt. % or less, and more preferably 97 wt. % or less.

In the case of using a luminescence dopant and an assist dopant, a content of the compound according to an aspect of the disclosure as a host molecule in the light-emitting layer is preferably 50 wt. % or greater, and more preferably 70 wt. % or greater. Moreover, in the case of using a luminescence dopant and an assist dopant, a content of the compound according to an aspect of the disclosure as a host molecule in the light-emitting layer is preferably 95 wt. % or less, and more preferably 90 wt. % or less.

In the case of using the compound according to an aspect of the disclosure as an exciplex molecule, a content of the compound in the light-emitting layer is preferably 30 wt. % or greater, and more preferably 40 wt. % or greater. Moreover, in the case of using the compound according to an aspect of the disclosure as an exciplex molecule, a content of the compound in the light-emitting layer is preferably 70 wt. % or less, and more preferably 60 wt. % or less.

Moreover, in a case where compounds (1) to (3) are incorporated as a material for forming the hole transport layer 5, an effect of efficiently transporting holes injected from the anode electrode into the light-emitting layer can be expected owing to an excellent hole transport property due to a carbazole skeleton.

Moreover, in a case where compounds (1) to (3) are incorporated as a material for forming the electron transport layer 6, an effect of efficiently transporting electrons injected from the cathode electrode into the light-emitting layer can be expected owing to an excellent electron transport property due to a benzene ring skeleton having an electron-withdrawing cyano group and a fluoroalkyl group.

According to the organic EL element including the configuration as described above, since the above-described compound according to an aspect of the disclosure is used, the organic EL element having improved luminous efficiency can be obtained.

The organic electroluminescence element according to the present embodiment described above emits light by applying an electric field between the anode electrode and the cathode electrode of the element obtained and by energizing the element. In this case, when the light emission is light emission from the excited singlet state, light having a wavelength corresponding to energy in the excited singlet state is confirmed as fluorescence emission and delayed fluorescence emission. Since normal fluorescence emission has a lifetime shorter than a lifetime of the delayed fluorescence emission, a luminescence lifetime can be distinguished between fluorescence (prompt fluorescence) and delayed fluorescence. On the other hand, when the light emission is light emission from the excited triplet state, a wavelength corresponding to energy in the excited triplet state is confirmed as phosphorescence. In the compound according to an aspect of the disclosure, phosphorescence can hardly be observed at room temperature. Under extremely low temperature conditions such as conditions by liquid nitrogen, phosphorescence emission can be observed.

### Third Embodiment

### Display Device

A display device according to the present embodiment includes a first organic EL element configured to emit red light, a second organic EL element configured to emit green light, and a third organic EL element configured to emit blue light, and at least any one of the first organic EL element, the second organic EL element, and the third organic EL element is the organic EL element according to the second embodiment described above.

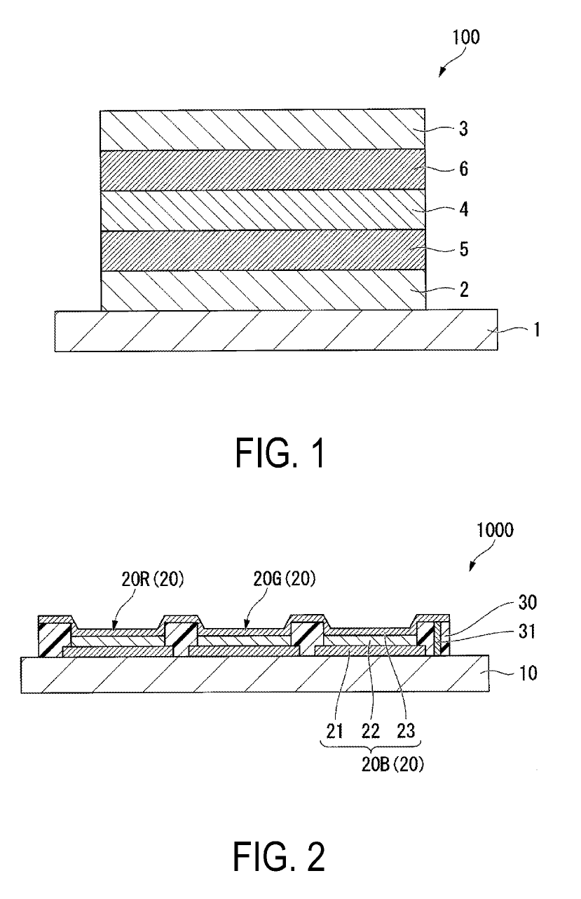

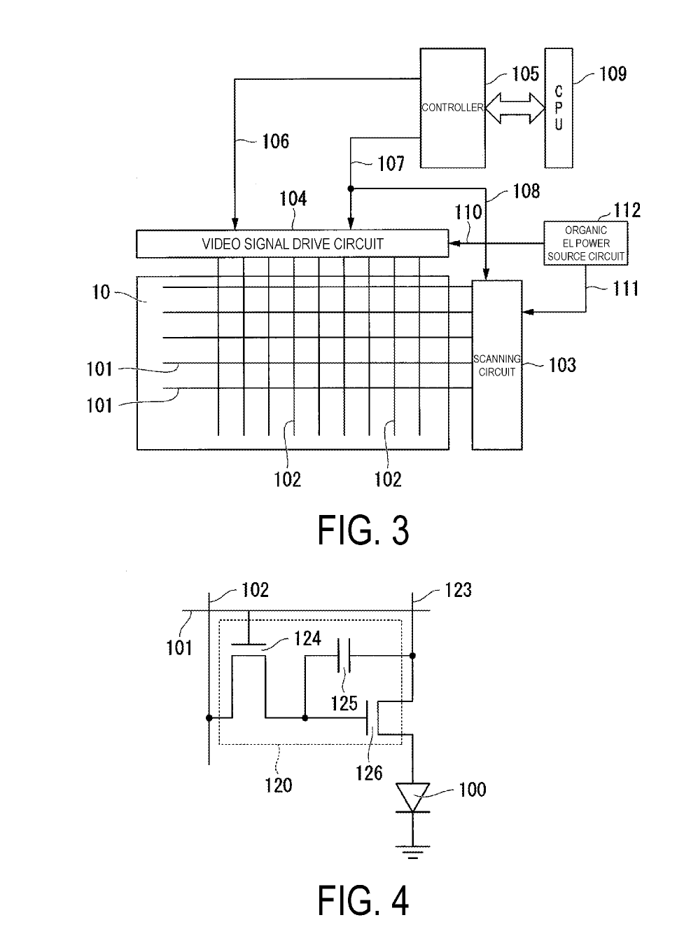

FIG. 2 is a schematic cross-sectional view of the display device according to the present embodiment. FIG. 3 is a plan view illustrating a main portion of the display device according to the present embodiment. FIG. 4 is an equivalent circuit diagram of one pixel illustrating a main portion of the display device according to the present embodiment. Note that in each embodiment described below, the same constituent elements as the constituent elements in the embodiments described above are denoted by the same reference signs, and detailed description of such constituent elements will be omitted.

As illustrated in FIG. 2, a display device 1000 includes an element substrate (substrate) 10, an organic EL element 20, and a partition 30. The display device 1000 according to the present embodiment includes a top-emitting type structure.

The element substrate 10 includes a pixel selection TFT element (not illustrated), a driving TFT element (not illustrated), and wiring lines (not illustrated) connected to these elements, respectively, on the substrate.

A plurality of the organic EL elements 20 are provided on the element substrate 10. The organic EL elements 20 each include a layered structure including a lower electrode 21, a light emitting portion 22, and an upper common electrode 23 layered one on another.

The lower electrode 21 is patterned for each of subpixels, and a plurality of the lower electrodes 21 are provided on the element substrate 10. The lower electrode 21 corresponds to the anode electrode 2 in the second embodiment.

The light emitting portion 22 includes a layered structure of a commonly known configuration including a hole injection layer, a hole transport layer, a light-emitting layer, an electron transport layer, an electron injection layer, and the like layered one on another. The light emitting portion 22 corresponds to the light-emitting layer 4, the hole transport layer 5, and the electron transport layer 6 in the second embodiment.

The upper common electrode 23 is provided across a plurality of the light emitting portions 22 to cover upper surfaces of the plurality of light emitting portions 22. The upper common electrode 23 corresponds to the cathode electrode 3 in the second embodiment.

The display device 1000 according to the present embodiment includes, as the organic EL element 20, an organic EL element (first organic EL element) 20R configured to emit red light, an organic EL element (second organic EL element) 20G configured to emit green light, and an organic EL element (third organic EL element) 20B configured to emit blue light.

The partition 30 partitions the plurality of lower electrodes 21 and the plurality of light emitting portions 22 for each of the subpixels. The upper common electrode 23 is provided to cover a top portion of the partition 30. Moreover, a through hole penetrating in a substrate thickness direction is provided inside the partition 30. A via 31 configured to connect a wiring line of the element substrate 10 and the upper common electrode 23 is provided inside the through hole.

In such a display device 1000, at least any one of the organic EL element 20R, the organic EL element 20G, and the organic EL element 20B is the organic EL element of the second embodiment described above. In the present embodiment, the organic EL element 20B is the organic EL element described in the second embodiment, and includes any of compounds (1) to (3) described above in a material for forming the light-emitting layer. A known material can be used for a material for forming each of other portions.

As illustrated in FIG. 3, in the display device according to an aspect of the disclosure, the element substrate 10 of the organic EL element is wired with scanning lines 101 and signal lines 102 in a matrix shape in a plan view. Each of the scanning lines 101 is connected to a scanning circuit 103 provided at an edge portion on one side of the element substrate 10. Each of the signal lines 102 is connected to a video signal drive circuit 104 provided at an edge portion on the other side of the element substrate 10. More specifically, a driving element such as a thin film transistor configured to drive the organic EL element is embedded in each of intersections of the scanning lines 101 and the signal lines 102, and a pixel electrode is connected for each of the driving elements. These pixel electrodes each correspond to the anode electrode 2 of the organic EL element.

The scanning circuit 103 and the video signal drive circuit 104 are electrically connected to a controller 105 via control lines 106, 107, and 108. The controller 105 is operated and controlled by a Central Processing Unit (CPU) 109. Moreover, an organic EL power source circuit 112 is separately connected to the scanning circuit 103 and the video signal drive circuit 104 via power source wiring lines 110 and 111. An image signal output unit includes the CPU 109 and the controller 105.

FIG. 4 is an equivalent circuit diagram constituting one pixel disposed in each of areas partitioned by the scanning lines 101 and the signal lines 102. In an area partitioned by the scanning line 101 and the signal line 102, a switching TFT 124, a holding capacitor 125, a TFT circuit 120 including a driving TFT 126, and the organic EL element 100 are provided. A power source line 123 is connected to the organic EL element 100.

In the pixel circuit illustrated in FIG. 4, when a scanning signal is applied to the scanning line 101, the scanning signal is applied to a gate electrode of the switching TFT 124 to turn the switching TFT 124 on.

Then, when a pixel signal is applied to the signal line 102, the pixel signal is applied to a source electrode of the switching TFT 124, and via the switching TFT 124 being on, electrically charges the holding capacitor 125 connected to a drain electrode of the switching TFT 124. The holding capacitor 125 is connected between the source electrode and the gate electrode of the driving TFT 126.

Therefore, a gate voltage of the driving TFT 126 is held at a value determined by a voltage of the holding capacitor 125 until the switching TFT 124 is scanned and selected next. The power source line 123 is connected to the organic EL power source circuit 112, and a current supplied from the power source line 123 flows to the organic EL element 100 via the driving TFT 126 to cause the organic EL element 100 to continuously emit light.

The display device according to an aspect of the disclosure uses compounds (1) to (3) described above in the organic EL element 100 and thus, becomes excellent in luminous efficiency.

### Fourth Embodiment

### Display Device

A display device according to the present embodiment includes the organic EL element according to the second embodiment described above and a phosphor layer configured to absorb light emitted from the organic EL element to fluoresce.

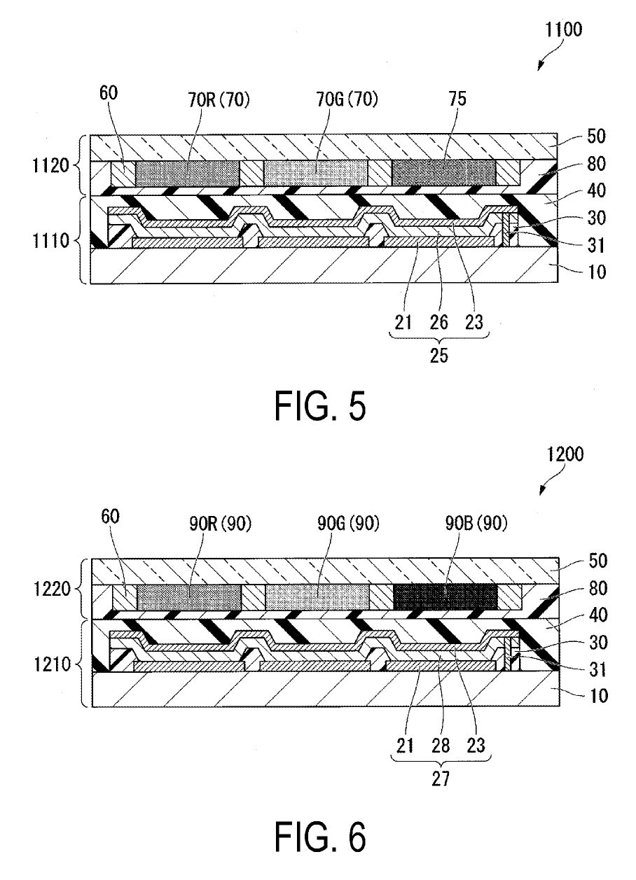

FIG. 5 is a schematic cross-sectional view of the display device according to the present embodiment. As illustrated in the figure, a display device 1100 according to the present embodiment includes an organic EL device 1110 and a wavelength conversion substrate 1120.

The organic EL device 1110 includes an element substrate (substrate) 10, an organic EL element 25, a partition 30, and a seal portion 40. The organic EL element 25 includes a layered structure including a plurality of lower electrodes 21, a light emitting portion 26 provided continuously to cover the plurality of lower electrodes 21, and an upper common electrode 23 provided continuously to cover the light emitting portion 26.

The organic EL device 1110 according to the present embodiment includes, as the organic EL element 25, the organic EL element described in the second embodiment and including any one of compounds (1) to (3) described above in a material for forming a light-emitting layer. The organic EL element 25 is configured to emit blue light by preparing a material for forming the light emitting portion 26 and the layered structure.

The seal portion 40 is provided on a surface of the element substrate 10 to cover a plurality of the organic EL elements 25 and a plurality of the partitions 30. The organic EL element 25 is generally vulnerable to oxygen and moisture, and readily deteriorates. The seal portion 40 has a function to protect the organic EL element 25 against oxygen and moisture.

A known material can be used as a material for forming the seal portion 40.

The wavelength conversion substrate 1120 is disposed on the seal portion 40 of the organic EL device 1110. The wavelength conversion substrate 1120 includes a substrate 50, a partition 60, a wavelength conversion layer (phosphor layer) 70, a light scattering layer 75, and a seal portion 80.

A transparent substrate can be used as the substrate 50. Examples of a material for forming the transparent substrate include an inorganic material such as glass, quartz glass, and silicon nitride, and a resin material such as an acrylic resin and a polycarbonate resin. Moreover, a substrate using a composite material formed by layering or mixing these materials can also be adopted, as long as the substrate has optical transparency. Moreover, a known flattened layer may be formed on a surface of the substrate 50 by using a resin material.

The partition 60 is provided in a lattice pattern by using a resin material, for example.

The wavelength conversion layer 70 includes a binder and a plurality of phosphor particles dispersed inside the binder. The wavelength conversion layer 70 is formed to be in contact with the partition 60.

The wavelength conversion layer 70 includes a wavelength conversion layer 70R configured to absorb excitation light and convert the light into red light to emit the red light, and a wavelength conversion layer 70G configured to absorb excitation light and convert the light into green light to emit the green light. The wavelength conversion layer 70R includes a red phosphor. The wavelength conversion layer 70G includes a green phosphor. As the binder, the red phosphor, and the green phosphor, a known binder, a known red phosphor, and a known green phosphor can be used.

The light scattering layer 75 can be configured to include a binder and a plurality of light scattering particles dispersed inside the binder. The light scattering layer 75 is formed to be in contact with the partition 60. As the binder and the light scattering particle, a known binder and a known light scattering particle can be used.

Moreover, the seal portion 80 is provided around the wavelength conversion layer 70 and between the wavelength conversion layer 70 and the organic EL device 1110. The seal portion 80 is configured to seal the partition 60, the wavelength conversion layer 70, and the light scattering layer 75. Moreover, the seal portion 80 is configured to bond the organic EL device 1110 to the wavelength conversion substrate 1120. As the seal portion 80, a known seal portion can be used.

In the display device 1100, the wavelength conversion layer 70 and the light scattering layer 75 are disposed to overlap, in a plan view, with each of the organic EL elements 25 (subpixels) of the organic EL device 1110.

Moreover, a circuit configuration around the display device 1100 can be the same as the circuit configurations illustrated in FIGS. 3 and 4 in the third embodiment.

In the display device 1100 including such a configuration, the blue light emitted from the organic EL element 25 is absorbed by the red phosphor of the wavelength conversion layer 70R to be converted into red light. Moreover, the blue light emitted from the organic EL element 25 is absorbed by the green phosphor of the wavelength conversion layer 70G to be converted into green light. Further, the blue light emitted from the organic EL element 25 is scattered by the light scattering layer 75. Accordingly, a color image can be displayed by using the red light, the green light, and the blue light.

Further, the display device 1100 according to the present embodiment uses the organic EL element according to an aspect of the disclosure as the organic EL element 25 and thus, becomes excellent in luminous efficiency.

### Fifth Embodiment

### Display Device

In the display device 1100 of the fourth embodiment, the organic EL device 1110 including the organic EL element according to an aspect of the disclosure emits the blue light, and the blue light emitted from the wavelength conversion substrate 1120 including the phosphor is converted into the green light and the red light and thus, a color image is displayed, but other configurations can also be adopted.

FIG. 6 is a schematic cross-sectional view of a display device according to the present embodiment. In an aspect of the disclosure, for example, as illustrated in FIG. 6, a display device 1200 may be configured to include an organic EL device 1210 and a color filter substrate 1220.

The organic EL device 1210 includes a plurality of organic EL elements 27. Each organic EL element 27 includes a layered structure including a plurality of lower electrodes 21, a light emitting portion 28 provided continuously to cover the plurality of lower electrodes 21, and an upper common electrode 23 provided continuously to cover the light emitting portion 28. The organic EL element 27 is configured to emit white light by preparing a material for forming the light emitting portion 28 and the layered structure. For example, the organic EL device 1210 according to the present embodiment corresponds to the illumination device according to an aspect of the disclosure.

The color filter substrate 1220 includes a substrate 50, a partition 60, a color filter layer 90, and a seal portion 80.

The color filter layer 90 includes a color filter layer 90R configured to partially absorb white light and transmit red light, a color filter layer 90G configured to partially absorb white light and transmit green light, and a color filter layer 90B configured to partially absorb white light and transmit blue light. In the display device 1200, the color filter layer 90 is disposed to overlap, in a plan view, with each of the organic EL elements 27 (subpixels) of the organic EL device 1210.

In the display device 1200 including such a configuration, the white light emitted from the organic EL element 27 is converted into red light by the color filter layer 90R. Moreover, the white light emitted from the organic EL element 27 is converted into green light by the color filter layer 90G. Further, the white light emitted from the organic EL element 27 is converted into blue light by the color filter layer 90B. Accordingly, a color image can be displayed by using the red light, the green light, and the blue light.

Further, the display device 1200 according to the present embodiment uses the organic EL element according to an aspect of the disclosure as the organic EL element 27 and thus, becomes excellent in luminous efficiency.

Note that as the illumination device according to an aspect of the disclosure is not limited to an illumination device such as the organic EL device 1210 according to the present embodiment including the plurality of lower electrodes 21 patterned for each of the subpixels, and the organic EL elements 27 corresponding to the lower electrodes 21 respectively and capable of emitting light. For example, as the illumination device according to an aspect of the disclosure, it is also possible to constitute an illumination device including a lower electrode larger (for example, one side has a unit of several centimeters) than the lower electrode 21 according to the present embodiment, and being usable as an indoor light source.

### Sixth Embodiment

### Electronic Device

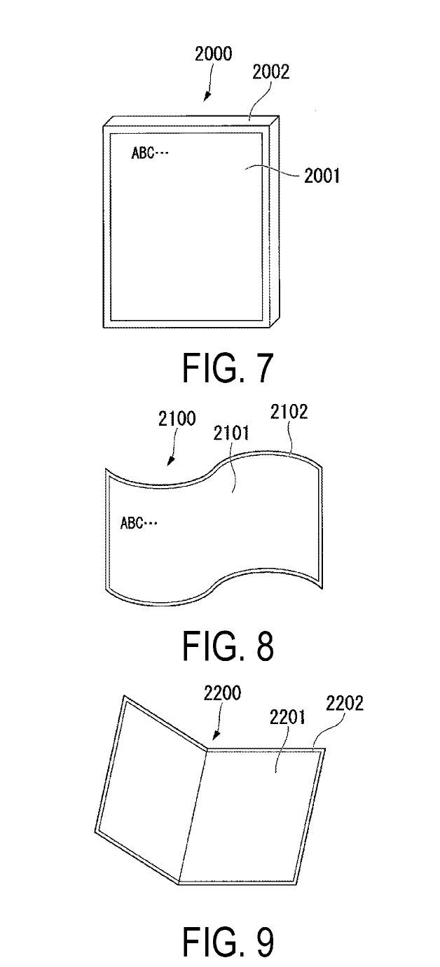

FIGS. 7 to 9 are schematic views illustrating electronic devices according to the present embodiment. The electronic devices according to the present embodiment each include the display device described above.

An electronic device 2000 illustrated in FIG. 7 includes the display device according to an aspect of the disclosure described above in a display portion 2001, and includes a battery, a communication module, and the like built in a main body portion 2002. Examples of the electronic device 2000 include, but not limited to, a smartphone, a tablet personal computer, an electronic book terminal, an electronic album, an electronic textbook, and an electronic dictionary. The electronic device 2000 uses the display device according to an aspect of the disclosure as the display portion 2001 and thus, can have extended use time as compared to the case where a display device including a configuration of the related art is used for a display portion.

An electronic device 2100 in another mode illustrated in FIG. 8 is a wearable display such as a tablet PC, a smartphone, and a clock, and includes a display portion 2101 and a main body portion 2102. The display portion 2101 adopts the display device according to an aspect of the disclosure described above. A flexible substrate (for example, a polymeric material) on which an organic EL element is layered is adopted in the display device and thus, the display portion 2101 can be bent, and mountability improves. Moreover, the electronic device 2100 uses the display device according to an aspect of the disclosure as the display portion 2101 and thus, can have extended use time as compared to the case where a display device including a configuration of the related art is used for a display portion.

An electronic device 2200 in another mode illustrated in FIG. 9 includes a display portion 2201 and a main body portion 2202. The display portion 2201 adopts the display device according to an aspect of the disclosure described above. A flexible substrate (for example, a polymeric material) on which an organic EL element is layered is adopted in the display device and thus, the display portion 2201 can be bent, and a foldable display is provided. The electronic device 2200 can be utilized as, for example, a pocketbook/paperback book type electronic book by taking advantage of being foldable. Moreover, the electronic device 2200 uses the display device according to an aspect of the disclosure as the display portion 2201 and thus, can have extended use time as compared to the case where a display device including a configuration of the related art is used for a display portion.

The preferred embodiments according to some aspects of the disclosure are described above with reference to the attached drawings, but it goes without saying that the disclosure is not limited to these examples. The shapes and the combinations of the constituent members described in the above examples are merely examples, and a variety of modifications can be made on the basis of design requirements or the like without departing from the gist of the disclosure.

### EXAMPLES

The disclosure will be described below in examples, but the disclosure is not limited to these examples. Note that in the examples described below, a unit of concentration “M” means “mol/L”.

Moreover, in the examples described below, each compound produced was subjected to measurement by the following apparatuses.

Absorption spectrum: “UV-2450” available from Shimadzu Corporation

Fluorescence spectrum and phosphorescence spectrum: “FluoroMax-4” available from Horiba, Ltd.

Photoluminescence quantum yield: “C9920-02G” available from Hamamatsu Photonics K.K.

Emission decay curve and time-resolved emission spectrum: “C11200” available from Hamamatsu Photonics K.K.

### Example 1

### Synthesis

A compound represented by the following formula (101) (hereinafter abbreviated as “compound (101)”) was produced in accordance with a procedure described below.

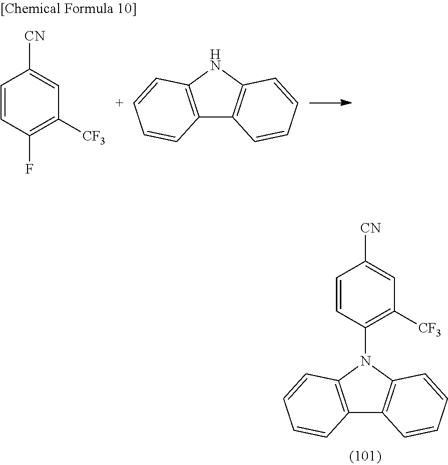 [SMILES: N#Cc1ccc(F)c(C(F)(F)F)c1 ; [C+]1=Cc2[nH]c3ccccc3c2C=C1 ; N#Cc1ccc(-n2c3ccccc3c3ccccc32)c(C(F)(F)F)c1]

A flask was charged with carbazole (22 g and 0.132 mol), 4-fluoro-3-(trifluoromethyl)benzonitrile (30 g and 0.159 mol), sodium tert-butoxide (17 g and 0.177 mol), and 1,3-dimethyl-2-imidazolidinone (DMI) (150 g), and the solution was stirred at room temperature for 3 hours. The resultant reaction liquid was added to 400 ml of cold water and then extracted with ethyl acetate.

An organic phase was washed with saturated brine and then dried over anhydrous magnesium sulfate. Further, an activated carbon (activated carbon powder, 037-02115; available from Wako Pure Chemical Industries, Ltd.) was added to the organic phase, and stirred for 5 minutes, and then the activated carbon was removed by filtration.

The resultant ethyl acetate solution was concentrated under reduced pressure and then crystallized with hexane to obtain crude crystals. The crude crystals were recrystallized with ethyl acetate/hexane to obtain white crystals of compound (101) (yield: 74.5%).

The resultant compound (101) was subjected to melting point measurement, MS measurement, and ¹H-NMR measurement.

According to the melting point measurement, a melting point was from 139 to 140° C.

Moreover, according to the MS measurement, a spectrum of m/z=336 was observed and data consistent with compound (101) was obtained.

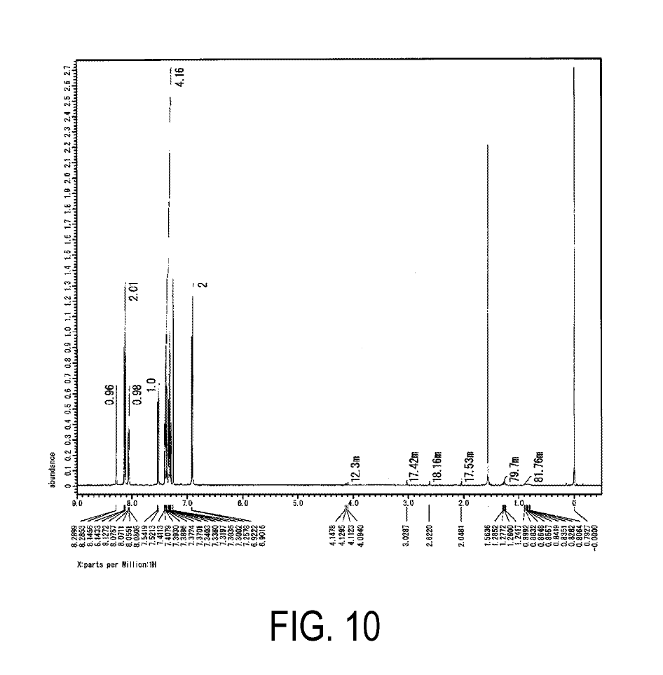

Moreover, according to the ¹H-NMR measurement, a spectrum specific to compound (101) was observed. FIG. 10 shows the ¹H-NMR spectrum.

From these results, it was confirmed that the resultant compound was compound (101) to be obtained.

### Evaluation of Physical Properties

Optical characteristics of the resultant compound (101) were measured. A solution sample was adjusted to have concentration of 5×10⁻⁵ mol/L by using a deoxygenated toluene solvent (oxygen concentration of 1 ppm or less; available from Wako Pure Chemical Industries, Ltd.) in a glove box filled with nitrogen gas (oxygen concentration of approximately 1 ppm and moisture concentration of approximately 0.1 ppm). Further, the resultant solution was placed in a quartz cell with a side-arm, and the solution in the quartz cell sealed with a septum cap was taken out of the glove box to be subjected to optical measurement.

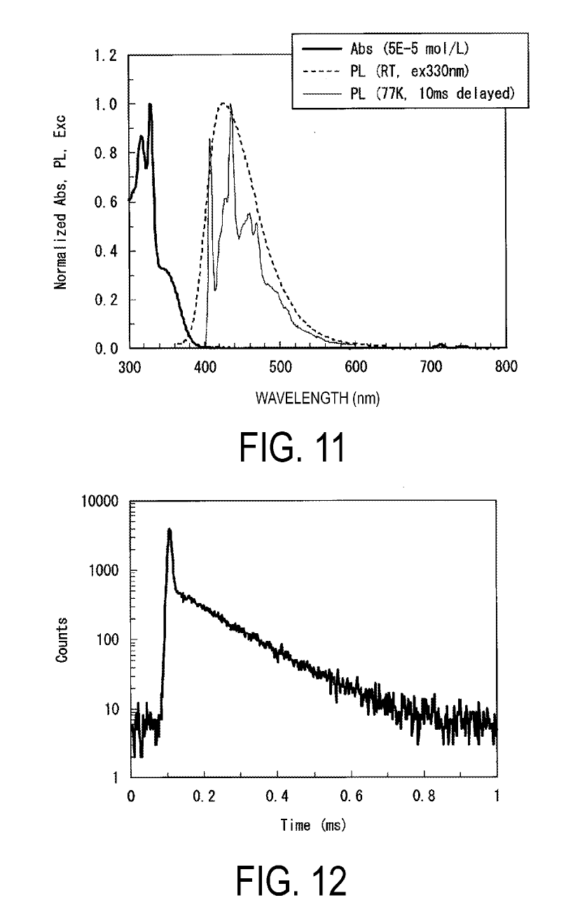

FIG. 11 shows an absorption spectrum, a fluorescence spectrum, and a phosphorescence spectrum of compound (101). A absorption maximum wavelength (λ_{Abs, max}) of compound (101) was 350 nm, a peak wavelength of fluorescence (λ_{PL, max}) was 424 nm, and a peak wavelength of phosphorescence (λ_{PL (77 K, 10 ms delayed), max}) was 408 nm.

Moreover, an energy level difference (ΔE_{ST}) between a singlet excited state (S₁) and a triplet excited state (T₁) of compound (101) was 0.14 eV. It is known that a smaller ΔE_{ST} more readily causes delayed fluorescence.

Further, a photoluminescence quantum yield (PLQY) of compound (101) was 0.56, and the compound was found to be a good luminescent material.

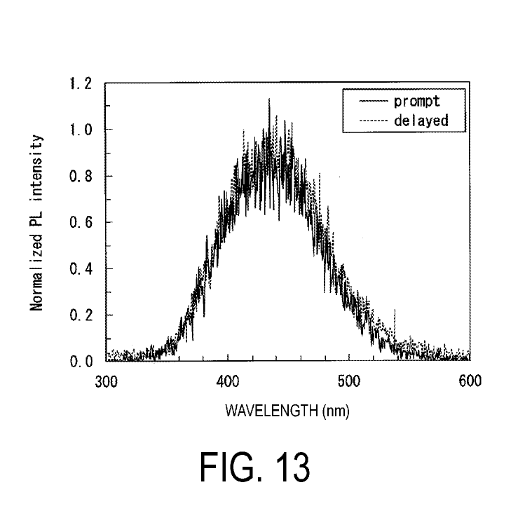

Moreover, an emission decay curve and a time-resolved emission spectrum of compound (101) were measured by using a solution sample having concentration of 1×10⁻⁴ mol/L. FIG. 12 shows the emission decay curve of compound (101). FIG. 13 shows the time-resolved emission spectrum of compound (101). FIG. 13 shows a prompt emission spectrum and a delayed emission spectrum.

The measurement in FIGS. 12 and 13 was conducted at room temperature. Moreover, in the measurement, ultraviolet light (337.1 nm) of a nitrogen gas laser was used as excitation light.

As found from FIG. 12, a component having the shortest luminescence lifetime was 9.3 ns and a component having the longest luminescence lifetime was 140 μs.

As shown in FIG. 13, the prompt emission spectrum of compound (101) exhibits fluorescence of the same spectral shape as the delayed emission spectrum of compound (101), and it is found that compound (101) causes delayed fluorescence.

As a result of analysis of FIG. 12, a TADF rate indicating a ratio of delayed fluorescence with respect to a total luminescence amount of fluorescence was found to be 0.69.

### Example 2

### Synthesis

A compound represented by the following formula (102) (hereinafter abbreviated as “compound (102)”) was produced in accordance with a procedure described below.

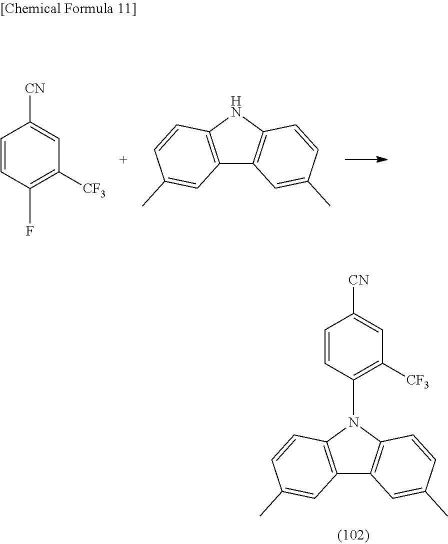

To a flask were added sodium hydride (60%, 15.5 g, 0.388 mol, and 1.5 eq) and DMF (350 mL), and the solution was stirred under a nitrogen atmosphere in the flask being cooled in an ice bath. To this suspension, a solution of 3,6-dimethylcarbazole (50.0 g and 0.256 mol) dissolved in DMF (350 mL) was added dropwise. After end of the dropwise addition, the ice bath was removed and the solution was stirred at room temperature for 2 hours.

Next, a solution of 4-fluoro-3-(trifluoromethyl)benzonitrile (58.0 g, 0.307 mol, and 1.2 eq) in DMF (350 mL) was added dropwise in the flask being cooled in a water bath, and after end of the dropwise addition, the solution was stirred at room temperature.

In 45 minutes after the start of the stirring at room temperature, disappearance of 3,6-dimethylcarbazole and production of compound (102) were confirmed by TLC and GC. The reaction was stopped by adding water (500 mL) and 1 N of hydrochloric acid (50 mL) to the reaction solution, and then compound (102) was extracted from the reaction solution with ethyl acetate (1 L×3 times and 300 mL'once).

An organic phase was washed with pure water (2 L×twice), and then washed with saturated brine (1 L), and the organic phase was dried over anhydrous magnesium sulfate (20.8 g), and then subjected to suction filtration. Filtrate was concentrated under reduced pressure to obtain 143 g of ocher powder.

The resultant ocher powder was purified with a silica gel column (1.22 kg of silica gel and developing solvent hexane/ethyl acetate=10:1) to obtain white powder (yield: 86.0 g and yield: 92.2%).

The 77.8 g of the obtained white powder was recrystallized with a mixed solvent of hexane/ethyl acetate (1.56 L of hexane and 0.117 L of ethyl acetate) to obtain white crystals of compound (102) (yield: 42 6 g and a recovery rate: 54.7%).

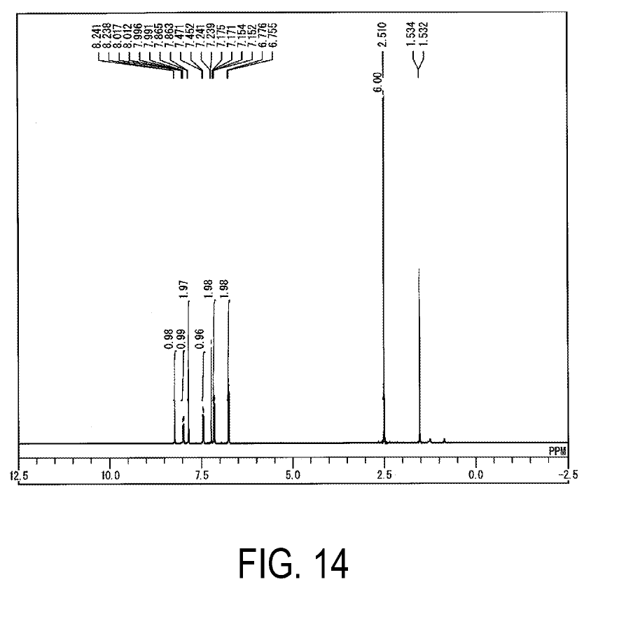

According to ¹H-NMR measurement of the resultant compound (102), a spectrum specific to compound (102) was observed. FIG. 14 shows the ¹H-NMR spectrum.

From this result, it was confirmed that the resultant compound was compound (102) to be obtained.

### Evaluation of Physical Properties

Optical characteristics of the resultant compound (102) were measured in the same manner as in Example 1.

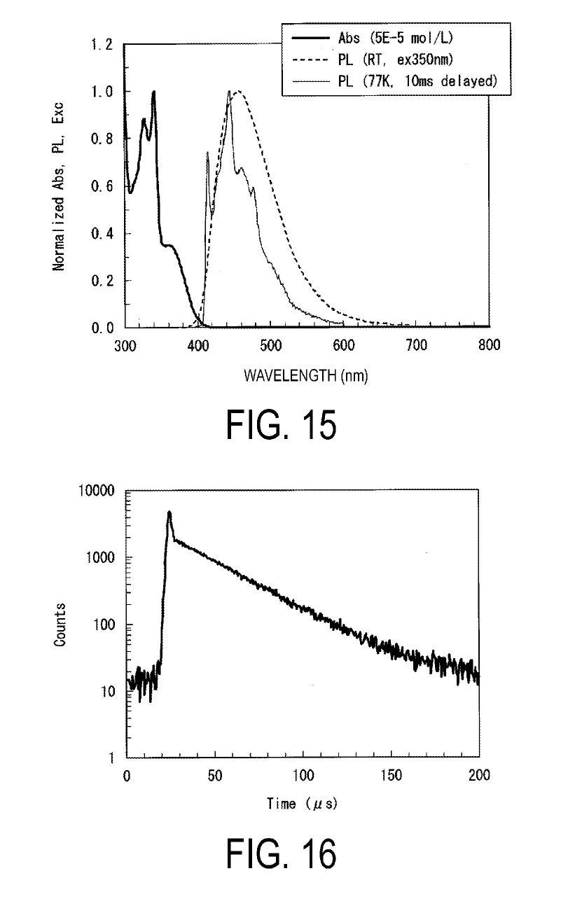

FIG. 15 shows an absorption spectrum, a fluorescence spectrum, and a phosphorescence spectrum of compound (102). An absorption maximum wavelength (λ_{Abs, max}) of compound (102) was 360 nm, a peak wavelength of fluorescence (λ_{PL, max}) was 457 nm, and a peak wavelength of phosphorescence (λ_{PL (77 K, 10 ms delayed), max}) was 415 nm.

Moreover, an energy level difference (ΔE_{ST}) between a singlet excited state (S₁) and a triplet excited state (T₁) of compound (102) was 0.07 eV.

Further, a photoluminescence quantum yield (PLQY) of compound (102) was 0.91, and was found to be significantly high.

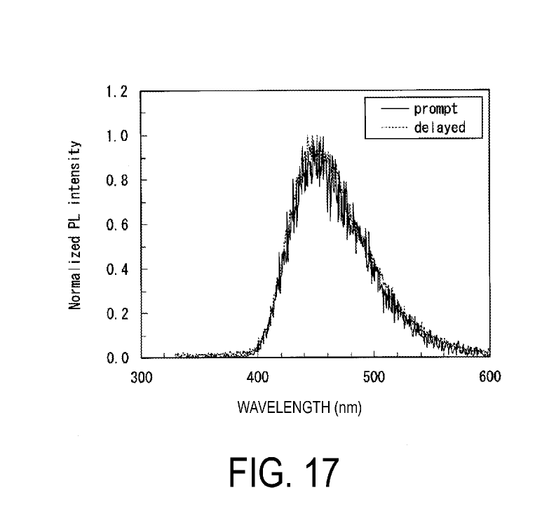

Moreover, an emission decay curve and a time-resolved emission spectrum of compound (102) were measured in the same manner as in Example 1 except that concentration of a solution sample was set to 5×10⁻⁵ mol/L. FIG. 16 shows the emission decay curve of compound (102). FIG. 17 shows the time-resolved emission spectrum of compound (102). FIG. 17 shows a prompt emission spectrum and a delayed emission spectrum.

As found from FIG. 16, a component having the shortest luminescence lifetime was 13.2 ns and a component having the longest luminescence lifetime was 31.1 μs.

As shown in FIG. 17, the prompt emission spectrum of compound (102) exhibits fluorescence of the same spectral shape as the delayed emission spectrum of compound (102), and it is found that compound (102) causes delayed fluorescence.

As a result of analysis of FIG. 16, a TADF rate indicating a ratio of delayed fluorescence with respect to a total luminescence amount of fluorescence was found to be 0.93.

### Example 3

### Synthesis

A compound represented by the following formula (103) (hereinafter abbreviated as “compound (103)”) was produced in accordance with a procedure described below.

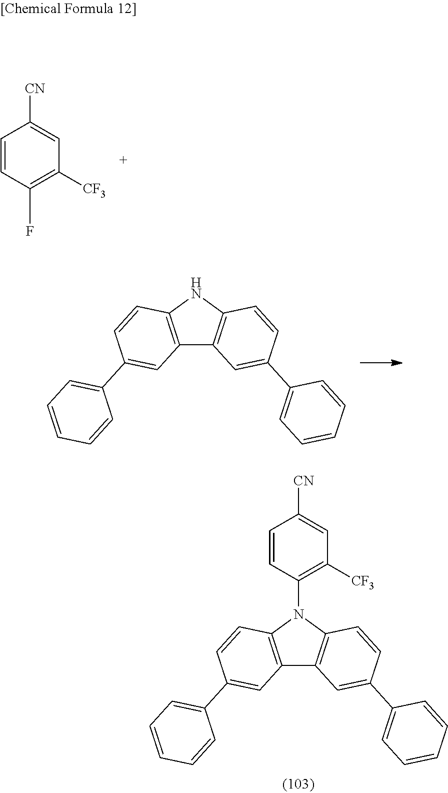

A flask was charged with 3,6-diphenylcarbazole (5 g and 15.6 mmol), 4-fluoro-3-(trifluoromethyl)benzonitrile (3.65 g and 19.3 mmol), sodium tert-butoxide (2.05 g and 21.3 mmol), and 1,3-dimethyl-2-imidazolidinone (DMI) (28 g), and the solution was stirred at room temperature (from 20 to 25° C.) for 3 hours. The resultant reaction liquid was added to 100 ml of cold water and then extracted with ethyl acetate.

An organic phase was washed with saturated brine and then dried over anhydrous magnesium sulfate. Further, an activated carbon (activated carbon powder, 037-02115; available from Wako Pure Chemical Industries, Ltd.) was added to the organic phase, and stirred for 5 minutes, and then the activated carbon was removed by filtration.

The resultant ethyl acetate solution was concentrated under reduced pressure and then 50 ml of warm ethanol was added to the solution to crystallize out. Ethyl acetate in the resultant crystal slurry was removed by azeotropic distillation with ethanol to obtain crude crystals. The resultant crude crystals were washed with ethanol and then air-dried to obtain white crystals of compound (103) (yield: 6.5 g and yield: 85%).

The resultant compound (103) was subjected to MS measurement and ¹H-NMR measurement.

According to the MS measurement, a spectrum of m/z=488 was observed, and data consistent with compound (103) was obtained.

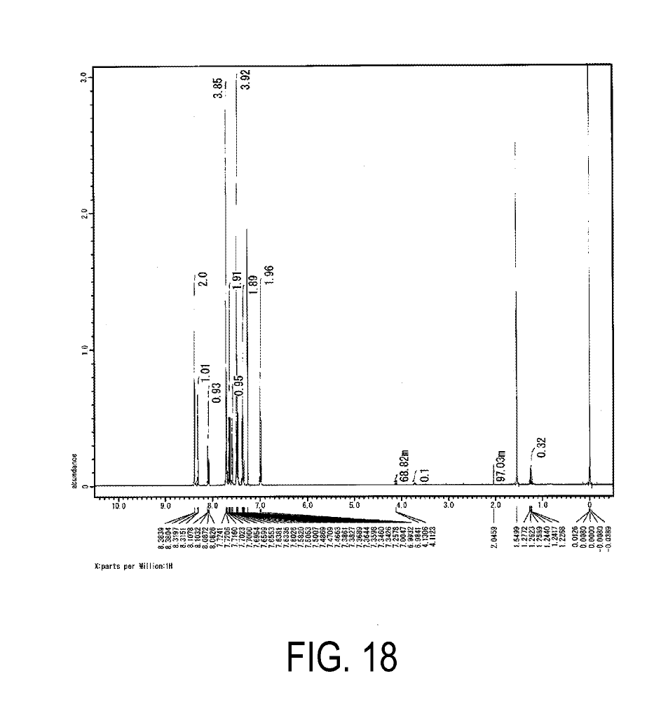

Moreover, according to the ¹H-NMR measurement, a spectrum specific to compound (103) was observed. FIG. 18 shows the ¹H-NMR spectrum.

From these results, it was confirmed that the resultant compound was compound (103) to be obtained.

### Evaluation of Physical Properties

Optical characteristics of the resultant compound (103) were measured in the same manner as in Example 1.

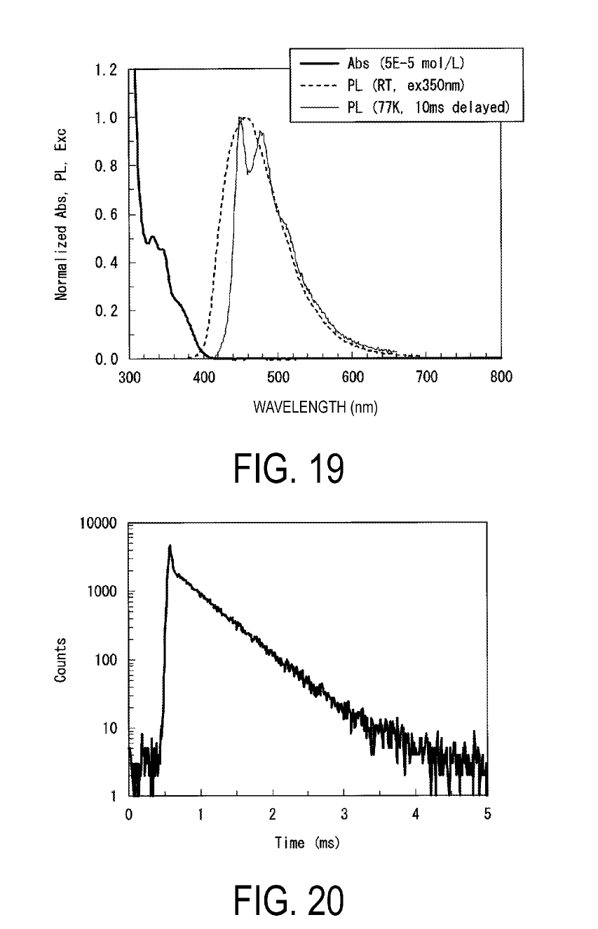

FIG. 19 shows an absorption spectrum, a fluorescence spectrum, and a phosphorescence spectrum of compound (103). An absorption maximum wavelength (λ_{Abs, max}) of compound (103) was 370 nm, a peak wavelength of fluorescence (λ_{PL, max}) was 457 nm, and a peak wavelength of phosphorescence (λ_{PL (77 K, 10 ms delayed), max}) was 449 nm.

Moreover, an energy level difference (ΔE_{ST}) between a singlet excited state (S₁) and a triplet excited state (T₁) of compound (103) was 0.20 eV.

Further, a photoluminescence quantum yield (PLQY) of compound (103) was 0.82, and was found to be high.

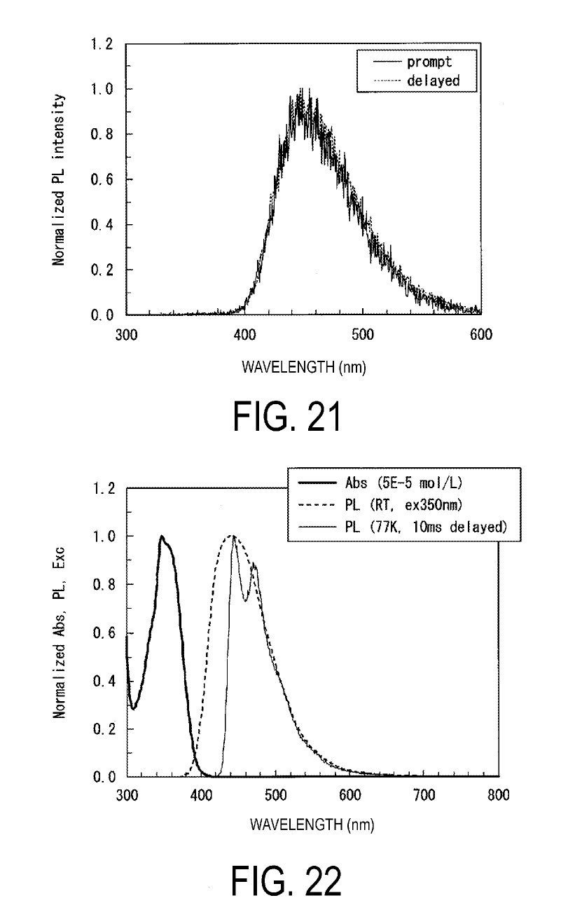

Moreover, an emission decay curve and a time-resolved emission spectrum of compound (103) were measured in the same manner as in Example 1 except that concentration of a solution sample was set to 5×10⁻⁵ mol/L. FIG. 20 shows the emission decay curve of compound (103). FIG. 21 shows the time-resolved emission spectrum of compound (103). FIG. 21 shows a prompt emission spectrum and a delayed emission spectrum.

As found from FIG. 20, a component having the shortest luminescence lifetime was 10.9 ns and a component having the longest luminescence lifetime was 503 μs.

As shown in FIG. 21, the prompt emission spectrum of compound (103) exhibits fluorescence of the same spectral shape as the delayed emission spectrum of compound (103), and it is found that compound (103) causes delayed fluorescence.

As a result of analysis of FIG. 20, a TADF rate indicating a ratio of delayed fluorescence relative to a total luminescence amount of fluorescence was found to be 0.91.

### Comparative Example 1

A compound represented by the following formula (201) (hereinafter abbreviated as “compound (201)”) was produced in accordance with a procedure described below.

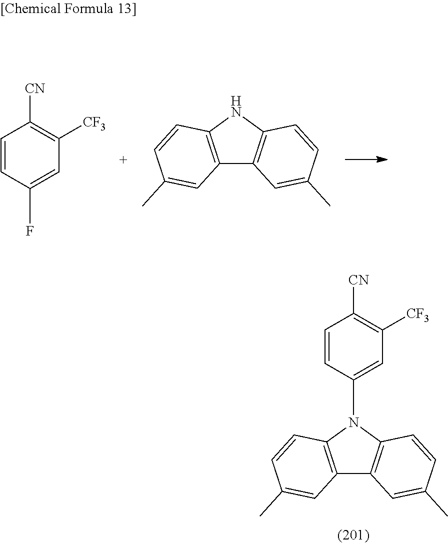

A flask was charged with 3,6-dimethylcarbazole (1 g and 5.1 mmol), 4-fluoro-2-(trifluoromethyl)benzonitrile (1.15 g and 5.9 mmol), sodium tert-butoxide (0.6 g and 6.2 mmol), and 1,3-dimethyl-2-imidazolidinone (DMI) (15 g), and the solution was stirred at room temperature (from 20 to 25° C.) for 3 hours. The resultant reaction liquid was cooled to 10° C. and then added to 50 ml of cold water, and then crystals were subjected to filtration.

The crystals separated by the filtration were dissolved in ethyl acetate, washed with water and saturated brine, and dried over anhydrous magnesium sulfate. An activated carbon (activated carbon powder, 037-02115; available from Wako Pure Chemical Industries, Ltd.) was added to the obtained ethyl acetate solution, and stirred for 5 minutes, and then the activated carbon was removed by filtration.

The resultant ethyl acetate solution was concentrated under reduced pressure and then crystallized by adding hexane. The resultant crystals were separated by the filtration and air-dried to obtain white crystals of compound (201) (yield: 86%).

According to MS measurement of the resultant compound (201), a spectrum of m/z=364 was observed, and data consistent with compound (201) was obtained. From this result, it was confirmed that the resultant compound was compound (201) to be obtained.

### Evaluation of Physical Properties

Optical characteristics of the resultant compound (201) were measured in the same manner as in Example 1.

FIG. 22 shows an absorption spectrum, a fluorescence spectrum, and a phosphorescence spectrum of compound (201). An absorption maximum wavelength (λ_{Abs, max}) of compound (201) was 356 nm, a peak wavelength of fluorescence (λ_{PL, max}) was 439 nm, and a peak wavelength of phosphorescence (λ_{PL (77 K, 10 ms delayed), max}) was 444 nm.

Moreover, an energy level difference (ΔE_{ST}) between a singlet excited state (S₁) and a triplet excited state (T₁) of compound (201) was 0.30 eV.

Further, a photoluminescence quantum yield (PLQY) of compound (201) was 0.49.

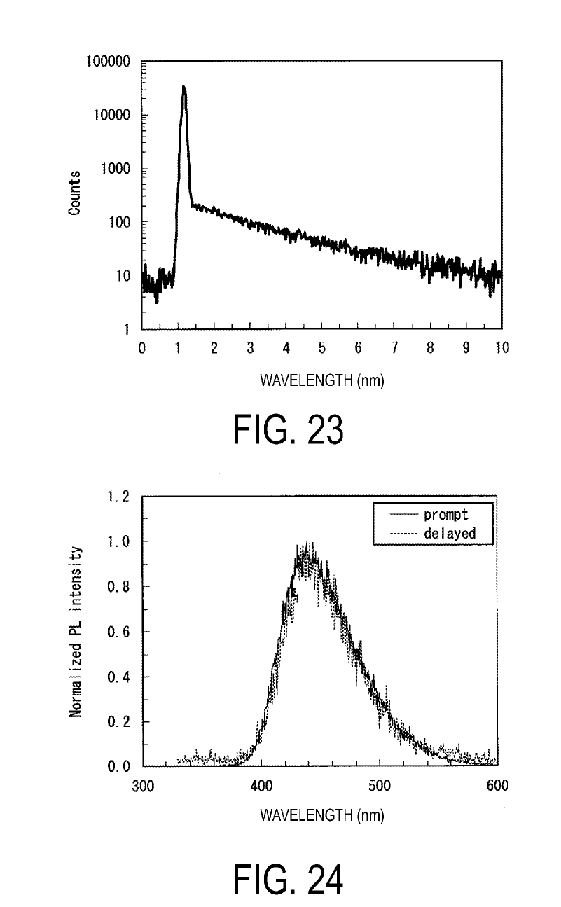

Moreover, an emission decay curve and a time-resolved emission spectrum of compound (201) were measured in the same manner as in Example 1 except that concentration of a solution sample was set to 5×10⁻⁵ mol/L. FIG. 23 shows the emission decay curve of compound (201). FIG. 24 shows the time-resolved emission spectrum of compound (201). FIG. 24 shows a prompt emission spectrum and a delayed emission spectrum.

As found from FIG. 23, a component having the shortest luminescence lifetime was 14.2 ns and a component having the longest luminescence lifetime was 2.28 μs.

As shown in FIG. 24, the prompt emission spectrum of compound (201) exhibits fluorescence of the same spectral shape as the delayed emission spectrum of compound (201), and it is found that compound (201) causes delayed fluorescence.

As a result of analysis of FIG. 23, a TADF rate indicating a ratio of delayed fluorescence relative to a total luminescence amount of fluorescence was found to be 0.12. It was found that compound (201) included almost no delayed fluorescence, and an effect of improving luminous efficiency was not expected very much when compound (201) was used as a luminescence dopant of an organic EL element.

### Example 4

A glass substrate (ITO substrate) having a thickness of 0.7 mm on which a anode electrode including an Indium Tin Oxide (ITO) having a film thickness of 100 nm was formed was washed with water, and then ultrasonically washed in an alkaline aqueous solution for 30 minutes. The substrate was washed with water, and further ultrasonically washed with ultrapure water for 15 minutes, and then dried at 110° C. for 30 minutes. Then, the resultant ITO substrate was subjected to UV-ozone treatment using a UV ozone cleaner in an air atmosphere.

The ITO substrate was set in a vacuum vapor deposition machine, and respective thin films were layered one on another on the ITO film as described below by a vacuum vapor deposition method. A degree of vacuum during the film formation was 5.0×10⁻⁵ Pa.

First, α-NPD (described below) was formed to have a thickness of 30 nm on the ITO film, TCTA (described below) was formed to have a thickness of 20 nm on the α-NPD, and further, CzSi (described below) was formed to have a thickness of 10 nm on the TCTA.

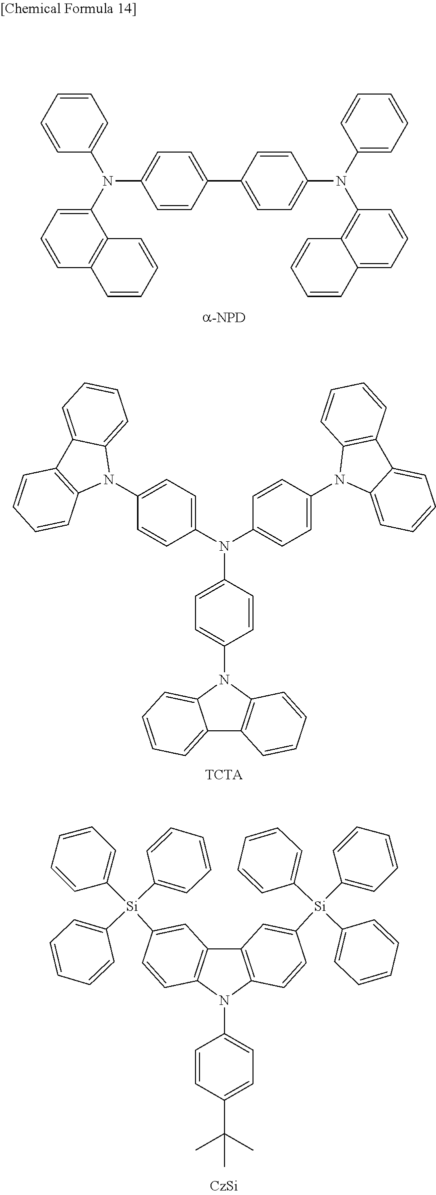 [SMILES: c1ccc(N(c2ccc(-c3ccc(N(c4ccccc4)c4cccc5ccccc45)cc3)cc2)c2cccc3ccccc23)cc1 ; c1ccc2c(c1)c1ccccc1n2-c1ccc(N(c2ccc(-n3c4ccccc4c4ccccc43)cc2)c2ccc(-n3c4ccccc4c4ccccc43)cc2)cc1 ; CC(C)(C)c1ccc(-n2c3ccc([Si](c4ccccc4)(c4ccccc4)c4ccccc4)cc3c3cc([Si](c4ccccc4)(c4ccccc4)c4ccccc4)ccc32)cc1]

Then, compound (101) and DPEPO (described below) were co-vapor-deposited from different vapor deposition sources to form a layer having a thickness of 20 nm to constitute a light-emitting layer. At this time, concentration of compound (101) was set to 10.0 wt. %.

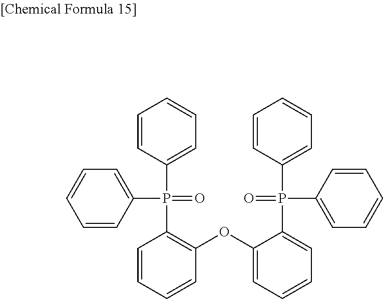 [SMILES: O=P(c1ccccc1)(c1ccccc1)c1ccccc1Oc1ccccc1P(=O)(c1ccccc1)c1ccccc1]

Then, DPEPO was formed to have a thickness of 10 nm, and TPBi (described below) was formed to have a thickness of 30 nm on the DPEPO. Further, lithium fluoride (LiF) was vapor-deposited to have a thickness of 0.5 nm, and then aluminum (Al) was vapor-deposited to have a thickness of 100 nm to form a cathode electrode.

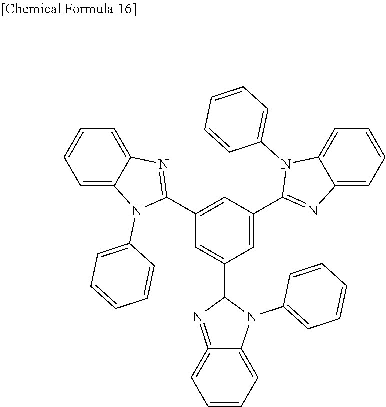

Then, the ITO substrate was bonded to the glass substrate with a photo-curable resin in a state where the ITO substrate and the glass substrate opposed each other to sandwich the layered structure prepared and thus, the layered structure was sealed and an organic EL element in Example 4 was prepared.

The organic EL element prepared was subjected to measurement using a source meter (model 2400, available from Keithley Instruments, Inc.) and a high-sensitivity spectral radiance meter (HS-1000, available from Otsuka Electronics Co., Ltd.).

In the organic EL element using compound (101) as a luminescent material, light emission having a peak wavelength of 430 nm was observed, and external quantum efficiency of 12% was obtained.

### Example 5

An organic EL element of Example 5 was prepared in the same manner as in Example 4 except that compound (102) was used in preparing a light-emitting layer.

As a result of measurement in the same manner as in Example 4, in the organic EL element using compound (102) as a luminescent material, light emission having a peak wavelength of 455 nm was observed, and external quantum efficiency of 15% was obtained.

### Example 6

An organic EL element of Example 6 was prepared in the same manner as in Example 4 except that compound (103) was used in preparing a light-emitting layer and that concentration of compound (103) was set to 12.0 mass %.

As a result of measurement in the same manner as in Example 4, in the organic EL element using compound (103) as a luminescent material, light emission having a peak wavelength of 456 nm was observed, and external quantum efficiency of 16% was obtained.

From the above results, it was found that the examples of the disclosure were useful.

### INDUSTRIAL APPLICABILITY

Some aspects of the disclosure can be applied, for example, to a novel compound improving luminous efficiency and capable of producing a high-performance organic EL element, and to an organic EL element, a display device, and an illumination device each using such a compound and having improved luminous efficiency.

### REFERENCE SIGNS LIST

1 Substrate
2 Anode electrode
3 Cathode electrode
4 Light-emitting layer
5 Hole transport layer
6 Electron transport layer
20, 20B, 20G, 20R, 25, 100 Organic EL element
70, 70G, 70R Wavelength conversion layer (Phosphor layer)
1000, 1100, 1200 Display device
1210 Organic EL device (Illumination device)
1220 Color filter substrate

1. A compound represented by general formula (1): 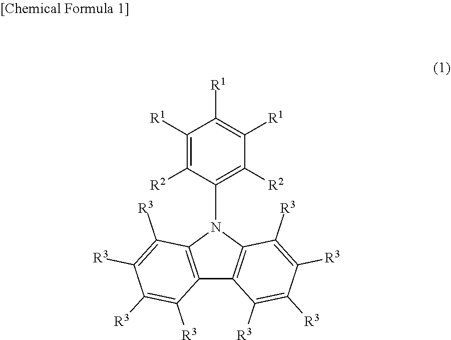
where R¹ is a hydrogen atom, a fluorine atom, an alkyl group having from 1 to 22 carbons, an alkoxy group having from 1 to 22 carbons, a fluorinated alkyl group having from 1 to 22 carbons, a cyano group, or a triphenylsilyl group, plural R¹ moieties are optionally the same as or different from one another, and only any one of the plural R¹ moieties is a cyano group,
R² is a hydrogen atom, a fluorine atom, an alkyl group having from 1 to 22 carbons, an alkoxy group having from 1 to 22 carbons, or a fluorinated alkyl group having from 1 to 22 carbons, plural R² moieties are optionally the same as or different from one another, and at least one of the R² moieties is a fluorinated alkyl group having from 1 to 22 carbons, and
R³ is a hydrogen atom, a fluorine atom, an alkyl group having from 1 to 22 carbons, an alkoxy group having from 1 to 22 carbons, a phenyl group, a carbazole group, a diphenylamino group, or a triphenylsilyl group, and plural R³ moieties are optionally the same as or different from one another.

2. The compound according to claim 1, represented by general formula (2): 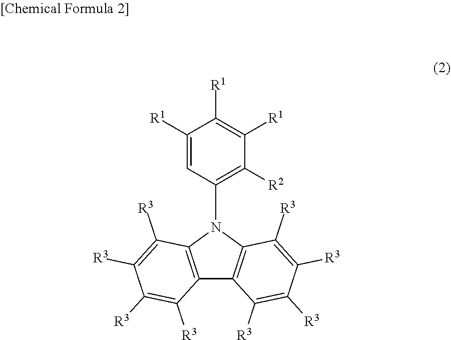
where R¹ and R³ are the same as in formula (1), and
R² is a fluorinated alkyl group having from 1 to 22 carbons.

3. The compound according to claim 2, represented by general formula (3): 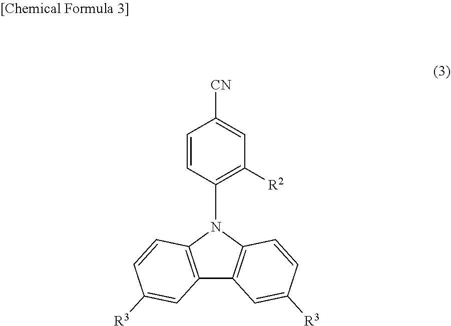
where R² is a fluorinated alkyl group having from 1 to 22 carbons, and
R³ is the same as in formula (1).

4. An organic EL element comprising a layer including the compound according to claim 1.

5. The organic EL element according to claim 4, comprising a light-emitting layer as the layer,
wherein the compound functions as a luminescence dopant.

6. The organic EL element according to claim 5, configured to emit delayed fluorescence.

7. The organic EL element according to claim 4, comprising a light-emitting layer as the layer,
wherein the compound functions as an assist dopant.

8. The organic EL element according to claim 4, comprising a light-emitting layer as the layer,
wherein the compound functions as a host compound.

9. The organic EL element according to claim 4, comprising a light-emitting layer as the layer,
wherein the compound functions as an exciplex molecule.

10. The organic EL element according to claim 4, comprising a hole transport layer as the layer.

11. The organic EL element according to claim 4, comprising an electron transport layer as the layer.

12. A display device comprising:
a first organic EL element configured to emit red light;
a second organic EL element configured to emit green light; and
a third organic EL element configured to emit blue light,
wherein at least any one of the first organic EL element, the second organic EL element, and the third organic EL element is the organic EL element according to claim 4.

13. A display device comprising:
the organic EL element according to claim 4; and
a phosphor layer configured to absorb light emitted from the organic EL element to fluoresce.

14. A display device comprising:
the organic EL element according to claim 4; and
a color filter layer configured to convert white light emitted from the organic EL element into at least one kind of red light, green light, and blue light.

15. An illumination device comprising the organic EL element according to claim 4 and configured to emit white light.
